(19) United States
(12) Patent Application Publication
(10) Pub. No.: US 2024/0299604 A1
(43) Pub. Date: Sep. 12, 2024

(54) SELF-SANITIZING HANDRAILS AND ASSOCIATED METHODS

(71) Applicant: RLW DESIGNS, LLC, Tomball, TX (US)

(72) Inventors: Ryan Matthew Ward, Tomball, TX (US); Laura Elizabeth Ward, Tomball, TX (US); Darrell Lessley Ward, Montgomery, TX (US)

(73) Assignee: RLW DESIGNS, LLC, Tomball, TX (US)

(21) Appl. No.: 18/270,968

(22) PCT Filed: Jan. 6, 2022
(86) PCT No.: PCT/US2022/011502

§ 371 Date: Jul. 5, 2023

Related U.S. Application Data
(60) Provisional application No. 63/134,400, filed on Jan. 6, 2021.

(51) Int. Cl. A61L 2/10; A61L 2/26; E04F 11/18; E05B 1/00

(52) U.S. Cl. CPC A61L 2/10; A61L 2/26; E04F 11/18; E05B 1/0069; A61L 2202/11; A61L 2202/14; E04F 2011/1868

(57) ABSTRACT

A self-sanitizing handrail system includes an elongate handle extending along a central axis and including an exterior surface graspable by users of the handrail system, an annular sanitizing module positioned around the handle and including an outer housing and a sanitizer positioned in the housing and configured to sanitize by a sanitizing light emitted by the sanitizer pathogens located on the exterior surface of the handle, a linear drive extending along the central axis of the handle and coupled to the sanitizing module, and a motor coupled to the linear drive and configured to transport the sanitizing module rectilinearly along the central axis of the handle in response to activation of the motor.

### CROSS-REFERENCE TO RELATED APPLICATIONS

The present application claims benefit of U.S. provisional patent application No. 63/134,400 filed Jan. 6, 2021, entitled “Self-Sanitizing Hand Rail,” which are incorporated herein by reference in their entirety for all purposes.

### STATEMENT REGARDING FEDERALLY SPONSORED RESEARCH OR DEVELOPMENT

Not applicable.

### BACKGROUND

Handrails are utilized in public and private settings for a variety of purposes. For examples, doors may incorporate handrails to be gripped by users for opening and closing the door. Handrails may also be positioned along stair cases to provide support for users travelling along the stair case. As one further example, handrails may be used as a barrier such as a safety barrier or other barrier which create an area of limited access to the public. Handrails, particularly those used in public settings, may be contacted by a large number of people over a given day. Viral and/or biological pathogens may be exchanged between an infected person's hand and an exterior surface of the handrail. Unless sanitized, the pathogens may reside on the exterior surface of the handrail for extended period of time during which the pathogens may be transferred to various other people who come into contact with the handrail. In this manner, pathogens may be exchanged from one user of the handrail to other users via contact with the pathogen-bearing exterior surface of the handrail.

### SUMMARY

An embodiment of a self-sanitizing handrail system includes an elongate handle extending along a central axis and comprising an exterior surface graspable by users of the handrail system, an annular sanitizing module positioned around the handle and comprising an outer housing and a sanitizer positioned in the housing and configured to sanitize by a sanitizing light emitted by the sanitizer pathogens located on the exterior surface of the handle, and a linear drive extending along the central axis of the handle and coupled to the sanitizing module, and a motor coupled to the linear drive and configured to transport the sanitizing module rectilinearly along the central axis of the handle in response to activation of the motor. In some embodiments, the linear drive comprises a lead screw rotatably coupled to the motor, and a travelling nut coupled to the sanitizing module and received within an interior passage of the handle, wherein the travelling nut and sanitizing module are each transportable linearly along the central axis of the handle in response to rotation of an output shaft of the motor. In certain embodiments, the handrail includes a control unit connected to a longitudinal end of the handle, wherein the control unit comprises an outer housing in which the motor is received, and wherein a longitudinal end of the lead screw extends into the outer housing of the control unit. In certain embodiments, the handle comprises an elongate slot which extends radially through the handle, and the sanitizing module comprises a mounting bracket coupled to the linear drive and which extends radially through the elongate slot of the handle. In some embodiments, the sanitizing module comprises an annular outer housing connected to a radially outer end of the mounting bracket, and an inner barrel received in an interior passage of the handle and coupled to a radially inner end of the mounting bracket, and wherein the sanitizer is received in the outer housing of the sanitizing module and extend arcuately around the exterior surface of the handle. In certain embodiments, the sanitizer of the sanitizing module includes one or more light modules configured to emit the sanitizing light directed against the exterior surface of the handle to sanitize pathogens located on the exterior surface

An embodiment of a self-sanitizing handrail system comprises an elongate handle extending along a central axis and comprising an exterior surface graspable by users of the handrail system, and an annular sanitizing module positioned around the handle and transportable linearly along the central axis of the handle, wherein the sanitizing module comprises a sanitizer including a light module configured to emit sanitizing light directed against the exterior surface of the handle to sanitize pathogens located on the exterior surface. In some embodiments, the sanitizing module comprise an annular outer housing which extends circumferentially around the exterior surface of the handle and forms a chamber in which the sanitizer is received, and wherein the sanitizer comprises an annular cradle which physically supports a plurality of the light modules which are circumferentially spaced about the exterior surface of the handle. In some embodiments, the light module comprises a printed circuit board (PCB) and a light emitting diode (LED) coupled to the PCB and configured to emit the sanitizing light directed against the exterior surface of the handle. In certain embodiments, the sanitizing module comprises a proximity sensor module configured to detect the presence of a person's hand in a path of travel of the sanitizing module. In some embodiments, the handrail includes a linear drive extending through an interior passage of the handle and coupled to the sanitizing module and configured to transport the sanitizing module rectilinearly along the central axis of the handle. In some embodiments, the handrail includes a control unit coupled to the handle and comprising a motor coupled to the sanitizing module, wherein the motor is configured to transport the sanitizing module linearly along the central axis of the handle, and a controller configured to control the activation of the motor. In certain embodiments, the controller comprises a sanitizer controller module configured to control the operation of the light module of the sanitizer.

An embodiment of a self-sanitizing handrail system comprises an elongate handle extending along a central axis and comprising an exterior surface graspable by users of the handrail system, an annular sanitizing module positioned around the handle and comprising an outer housing and a sanitizer positioned in the housing of the housing and configured to sanitize by a sanitizing light emitted by the sanitizer pathogens located on the exterior surface of the handle, and a control unit coupled to the handle and comprising a motor coupled to the sanitizing module, wherein the motor is configured to transport the sanitizing module linearly along the central axis of the handle in response to activation of the motor, and a controller configured to control the activation of the motor. In some embodiments, the handle comprises a touch sensor module positioned on the exterior surface of the handle and connected to the controller, and wherein the controller is configured to determine a longitudinal contact location along the handle at which a person's hand contacts the handle in response to the person's hand contacting the touch sensor module at the contact location. In some embodiments, the controller is configured to transport the sanitizing module to the contact location to sanitize the contact location in response to the person's hand contacting the touch sensor module at the contact location. In certain embodiments, the sanitizing module comprises a proximity sensor module that is connected to the controller and configured to detect the presence of a person's hand in a path of travel of the sanitizing module. In certain embodiments, the controller is configured to continuously transport the sanitizing module between a first position located at a first end of the handle, and a second position located at a second end of the handle longitudinally opposite the first end. In some embodiments, the handrail includes a linear drive extending through an interior passage of the handle and coupled to the sanitizing module and configured to transport the sanitizing module rectilinearly along the central axis of the handle. In some embodiments, the sanitizer of the sanitizing module includes one or more light modules configured to emit the sanitizing light directed against the exterior surface of the handle to sanitize pathogens located on the exterior surface.

### BRIEF DESCRIPTION OF THE DRAWINGS

For a detailed description of exemplary embodiments of the disclosure, reference will now be made to the accompanying drawings in which:

FIG. 1 is a perspective view of an embodiment of a door incorporating a self-sanitizing handrail;

FIG. 2 is a perspective view of an embodiment of a free-standing rail incorporating a self-sanitizing handrail;

FIG. 3 is a perspective view of an embodiment of a staircase incorporating a self-sanitizing handrail;

FIG. 4 is a perspective view of an embodiment of a double door incorporating a plurality of the handrails of FIG. 1;

FIGS. 5-7 are perspective views of an embodiment of a control unit of the handrail of FIG. 1;

FIG. 8 is a side view of the control unit of FIGS. 5-7;

FIG. 9 is a front view of the control unit of FIGS. 5-7;

FIG. 10 is a side cross-sectional view of the handrail of FIG. 1;

FIG. 11 is a zoomed-in side cross-sectional view of the handrail of FIG. 1;

FIG. 12 is a rear view of an embodiment of a handle assembly of the handrail of FIG. 1;

FIG. 13 is a perspective cross-sectional view of an embodiment of a sanitizing module of the handrail of FIG. 1;

FIG. 14 is a perspective view of an embodiment of a sanitizer of the sanitizing module of FIG. 13;

FIG. 15 is a top-down cross-sectional view of the handrail of FIG. 1;

FIG. 16 is a block diagram of an embodiment of a controller of the control unit of FIGS. 5-7;

FIG. 17 is a perspective view of another embodiment of a self-sanitizing handrail;

FIG. 18 is a side view of another embodiment of a self-sanitizing handrail;

FIG. 19 is a perspective view of the handrail of FIG. 18;

FIG. 20 is a partial perspective view of another embodiment of a self-sanitizing handrail;

FIG. 21 is a partial perspective view of another embodiment of a self-sanitizing handrail;

FIG. 22 is a perspective view of an embodiment of an internal support of the handrail of FIG. 21;

FIG. 23 is an end cross-sectional view of the handrail of FIG. 21; and

FIG. 24 is a perspective view of another embodiment of a self-sanitizing handrail.

### DETAILED DESCRIPTION

The following discussion is directed to various exemplary embodiments. However, one skilled in the art will understand that the examples disclosed herein have broad application, and that the discussion of any embodiment is meant only to be exemplary of that embodiment, and not intended to suggest that the scope of the disclosure, including the claims, is limited to that embodiment.

Certain terms are used throughout the following description and claims to refer to particular features or components. As one skilled in the art will appreciate, different persons may refer to the same feature or component by different names. This document does not intend to distinguish between components or features that differ in name but not function. The drawing figures are not necessarily to scale. Certain features and components herein may be shown exaggerated in scale or in somewhat schematic form and some details of conventional elements may not be shown in interest of clarity and conciseness.

In the following discussion and in the claims, the terms “including” and “comprising” are used in an open-ended fashion, and thus should be interpreted to mean “including, but not limited to . . . ” Also, the term “couple” or “couples” is intended to mean either an indirect or direct connection. Thus, if a first device couples to a second device, that connection may be through a direct connection, or through an indirect connection via other devices, components, and connections. In addition, as used herein, the terms “axial” and “axially” generally mean along or parallel to a central axis (e.g., central axis of a body or a port), while the terms “radial” and “radially” generally mean perpendicular to the central axis. For instance, an axial distance refers to a distance measured along or parallel to the central axis, and a radial distance means a distance measured perpendicular to the central axis. Any reference to “up,” “upper,” “upwardly,” “down,” “lower,” “downwardly” and the like in the description and the claims is made for purposes of clarity.

As described above, handrails may be utilized for a variety of purposes in private and public settings for, as an example, opening and closing doors, providing support to people travelling along a stair case, and for providing barriers which define an area of restricted access. Additionally, given that handrails may come into direct physical contact with a large number of people over a given day, viral and/or biological pathogens may be transferred from infected users of the handrail to an exterior surface thereof. These pathogens may reside on the handrail over an extended period of time and thereby be transferred to multiple other users of the handrail. In this manner, critical safety and support infrastructure like handrails may increase the spread and transmission of dangerous pathogens unless frequently and properly sanitized to ensure that any pathogens transferred to the handrail are promptly sanitized to ensure they are not spread to other users of the handrail. Indeed, one of the primary mechanisms for the spread of the SARS-CoV-2 virus (the virus responsible for the COVID-19 disease) has been physical contact between uninfected persons and surfaces (e.g., handrails, door handles, etc.) harboring the SARS-CoV-2 virus. Thus, promptly and properly sanitizing surfaces, such as handrails, which come into physical contact with large numbers of people may markedly reduce the communal spread of dangerous pathogens such as, for example, the SARS-CoV-2 virus.

In conventional practice, surfaces like handrails which are physically contacted by large numbers of people are manually sanitized at irregular or excessively long intervals which allows for pathogens to reside on the given surface for a long enough period of time before sanitization to spread to other people through physical contact. In many instances, it may simply be impractical or otherwise undesirable to sanitize a frequently contacted surface at intervals sufficient to minimize the spread of pathogens caused through physical contact with the surface.

In addition, door handles have been developed which may self-sanitize and thus avoid the need of cumbersome manual sanitization. Typically, self-sanitizing door handles include a reservoir of liquid sanitizer and a powered mechanism such as a pump for pumping the sanitizer through one or more nozzles for applying the sanitizer as droplets and/or as an aerosol to the exterior of the door handle. While such self-sanitizing door handles may provide advantages in at least some applications, the coverage provided by the one or more nozzles of the self-sanitizing door handle may be insufficient for large surfaces such as handrails, limiting such devices to relatively small surfaces like door handles. Additionally, self-sanitizing door handles require the periodic refilling of the liquid sanitizer as the sanitizer is consumed by the self-sanitizing door handle. The refilling of the liquid sanitizer is an additional expense for the operator of the self-sanitizing door handle (e.g., the owner of the building including the door handle). Through error or neglect the liquid sanitizer of the self-sanitizing door handle may not always be promptly refilled, preventing the door handle from being properly sanitized. Further, the liquid sanitizer when applied to the door handle may be unpleasant for users when they come into physical contact with the applied sanitizer.

Accordingly, embodiments of self-sanitizing handrails are described herein which include a sanitizing module transportable along a longitudinal length of a handle of the self-sanitizing handrail to automatically sanitize the handle thereof. The automatic sanitization provided by the sanitizing module may negate the requirement of an operator to frequently sanitize the handrail to mitigate the transmission of pathogens between users of the handrail. Additionally, the sanitizing module may travel along the longitudinal length of the handle, increasing the surface area which may be automatically sanitized relative to conventional self-sanitizing door handles which rely on a stationary sanitizer. Particularly, embodiments of handrails described herein include a linear drive connected to the sanitizing module and configured to displace the sanitizing module rectilinearly along a central axis of the handle. In this manner, the self-sanitizing handrails described herein may be utilized in a variety of applications including applications having relatively long handrails such as free-standing rails and the handrails of staircases.

Additionally, at least some of the embodiments of self-sanitizing handrails described herein include a sanitizer which sanitizes using a sanitizing, short-wavelength light emitted by one or more light modules of the sanitizer. For example, the light modules may be configured to emit light in the ultraviolet (UV) and/or blue-wavelengths less than approximately 450 nanometers (nm). As used herein, the term “sanitizing light” refers to light having a wavelength of less than 450 nm. The operation of the light modules as well as the linear drive of the handrail used to transport the sanitizing module may be controlled by a control unit of the self-sanitizing handrail. By utilizing electrically powered light modules for sterilization, the requirement for resupplying the sanitizing module with a liquid sanitizer may be avoided with the handrail requiring only electrical power for operation which may be delivered with a variety of means including, for example, an external power system, a rechargeable battery module, and/or a solar panel or array.

Embodiments of self-sanitizing handrails described herein may also include one or more sensors for providing sensor feedback to the controller of the handrail. For instance, the handrail may include one or more proximity sensor modules configured to detect the presence of a person's hand or other object located in a path of travel of the sanitizing module. Handrails disclosed herein may also include a touch sensor module coupled to the handle of the handrail and configured to detect contact with a person's hand other object. The controller may, based on sensor data provided by the touch sensor, determine a contact location along the longitudinal length of the handle at which the person's hand contacts the handle. The controller may rely on such sensor feedback in controlling the operation of the linear drive and/or sanitizer. For example, the controller may temporarily disable operation of the linear drive and/or sanitizer in response to the detection of a person's hand either contacting the handle or position in a path of travel of the sanitizing module.

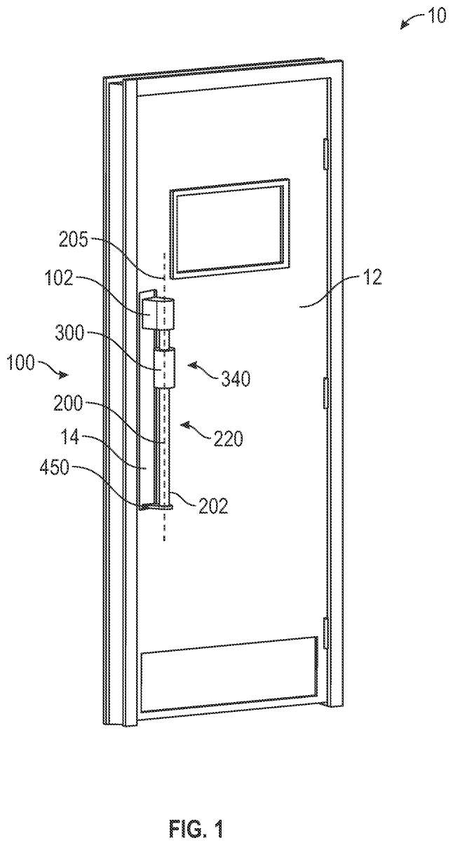

Referring initially to FIG. 1, an embodiment of a door incorporating a self-sanitizing handrail 100 is shown. Door 10 may be opened and closed by users to provide access to an indoor room or outdoor area and generally includes a door panel 12, a mounting plate 14 affixed to a surface of the door panel 12, and the self-sanitizing handrail 100 coupled to the mounting plate 14. It may be understood that the door 10 shown in FIG. 1 is only exemplary and the configuration of door 10 may vary substantially in different embodiments. As an example, while in this embodiment the handrail 100 is mounted to the door 12 using mounting plate 14, in other embodiments, door 10 may not include mounting plate 14 and instead handrail 100 may be mounted directly to the door panel 12. Additionally, it may be noted that handrail 100 may be incorporated in doors comprising various materials including metal doors, wooden doors, glass doors, etc. Further, while door 10 is shown in FIG. 1 as a door sized for the passage of people, in other embodiments, door 10 may comprise a smaller door such as a door of a cabinet or other structure.

In this exemplary embodiment, the handrail 100 of door 10 generally includes a control module 102, an elongate handle assembly 200 extending from the control module 102, a sanitizing module 300 positioned along the handle assembly 200, and a handle bracket 450 coupled to an end of the handle assembly 200. As will be discussed further herein, control module 102 powers and controls the operation of both handle assembly 200 and sanitizing module 300. Additionally, in this exemplary embodiment, control module 102 acts as a bracket which, along with handle bracket 450, couples (e.g., via one or more fasteners extending through module 102 and handle bracket 450 and the corresponding mounting plate 14) the handrail 100 to the mounting plate 14 of door 10.

Handle assembly 200 of the handrail 100 is configured to be physically contacted by users of the door 10 such that the users may open and close the door 10. Handle assembly 200 has a central or longitudinal axis 205 and an outer surface or exterior surface 202 exposed to the surrounding environment. In this exemplary embodiment, and as will be discussed further herein, handle assembly 200 includes a linear drive 220 mechanically coupled between the control module 102 and sanitizing module 300 and which transports the sanitizing module 300 along handle assembly 200. In this exemplary embodiment, the linear drive 220 of handle assembly 200 is powered and controlled by the control module 102. Linear drive 220 transports sanitizing module 300 by mechanically transferring motion from control module 102 to the sanitizing module 300. Additionally, while in this exemplary embodiment linear drive 220 of handle assembly 200 is incorporated into handle assembly 200, in other embodiments, linear drive 220 may be incorporated into sanitizing module 300 instead of handle assembly 200. For example, sanitizing module 300 may comprise an actuator powered by control module 102 and configured to transport sanitizing module 300 along handle assembly 200.

Sanitizing module 300 is configured to selectably sanitize the handle assembly 200 as controlled by the control module 102. Particularly, sanitizing module 300 includes a sanitizer 340 which sanitizes the exterior surface 202 of handle assembly 200. As will be described further herein, sanitizer 340 may comprise light-based sanitizer which sanitizes the exterior surface 202 of handle assembly 200 using sanitizing light. Sanitizer 340 may comprise, in lieu of or addition to the light-based sanitizer, a cloth or other flexible, absorbent member impregnated with a liquid sanitizer. Sanitizer 340 may sanitize the entire circumference or perimeter of the exterior surface 202 of handle assembly 200. Additionally, the absorbent member may wipe the exterior surface 202 of the exterior handle 201 to remove any debris which may hinder the operation of a light-based sanitizer of the sanitizing module 300.

Additionally, sanitizer 340 may sanitize substantially the entire longitudinal length of exterior surface 202 as sanitizing module 300 is transported along the handle assembly 200 by linear drive 220. Particularly, sanitizing module 300 may travel between a first outermost position directly adjacent or abutting the control module 102 and a second outermost position directly adjacent or abutting the handle bracket 450. It may be understood that sanitizing module 300 may travel to or from any position along the handle assembly 200 located between the first and second outermost positions as controlled by the control module 102. Given that sanitizing module 300 may travel along substantially the entire longitudinal length of handle assembly 200, sanitizing module 300 may sanitize the entire, or at least substantially the entire, surface area of the exterior surface 202 of handle assembly 200. Moreover, given that sanitizing module 300 may travel along the longitudinal length of handle assembly 200, the length of handle assembly 200 may vary substantially, encompassing applications beyond the door handle application illustrated in FIG. 1.

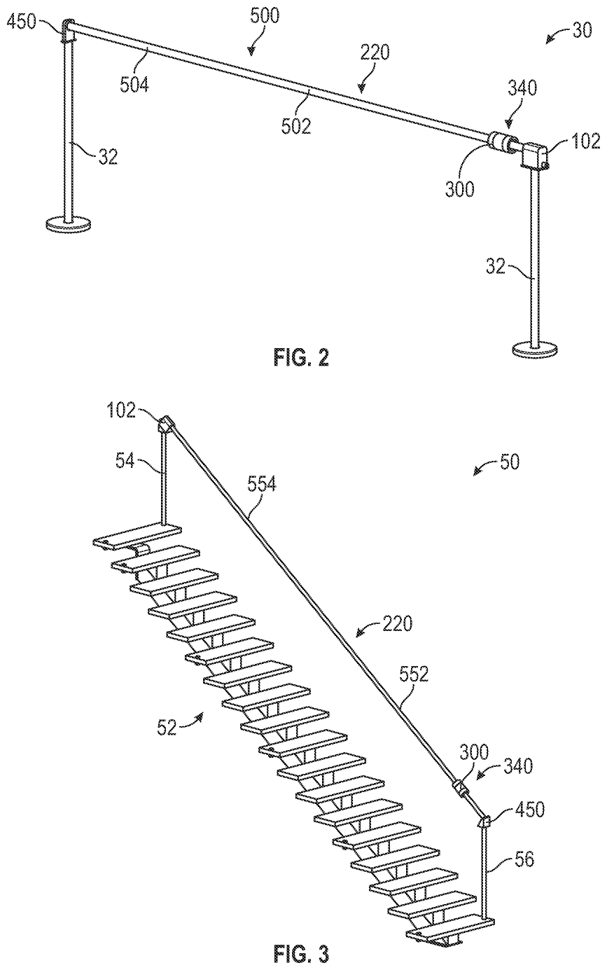

For example, referring now to FIG. 2, an embodiment of a free-standing rail or barrier 30 is shown including a self-sanitizing handrail 500. Free-standing rail 30 may be used as a barrier for restricting access to a particular area. Alternatively, free-standing rail 30 may comprise a safety feature providing a means for a user to physically support themselves as they walk alongside the free-standing rail 30. In this exemplary embodiment, free-standing rail 30 generally includes a pair of vertically extending support stands 32. One of the support stands 32 couples to the control module 102 while the other support sand 32 couples to the handle bracket 450 thereby positioning handrail 500 vertically above the floor upon which support stands 32 are positioned in a free-standing arrangement such that free-standing rail 30 is not coupled to or support by any external structure.

The handrail 500 is similar in configuration as the handrail 100 shown in FIG. 1 except that handrail 500 is coupled to support stands 32 and a handle 502 of handrail 500 has a greater longitudinal length than the handle 202 of handrail 100. As with handrail 100, the sanitizing module 300 of handrail 500 may travel across substantially the entire longitudinal length of handle 502 to ensure that at least the substantial entirety of the surface area of an outer surface or exterior 504 of handle 502 may be sanitized by sanitizing module 300.

Referring now to FIG. 3, an embodiment of a staircase 50 is shown which includes a self-sanitizing handrail 550. Staircase 50 generally includes a plurality of steps 52 at different vertical elevations, a pair of support stands 54, 56, and the handrail 550. Similar to support stands 32 shown in FIG. 2, the support stands 54, 56 of staircase 50 physically support and position the handrail 550. In this exemplary embodiment, a first support stand 54 extends from an uppermost step 52 of staircase 50 and connects to the control module 102 of handrail 550 while a second support stand 56 extends from a lowermost step 52 of staircase 50 and connects to the handle bracket 450 of handrail 550. In this arrangement, handrail 550 extends at a vertical incline parallel with the steps 52 of staircase 50. It may be understood that the staircase 50 shown in FIG. 3 is only exemplary and the configuration of staircase 50 may vary in other embodiments. For example, in other embodiments, rather than being supported by support stands 54, 56, handrail 550 may be mounted to a vertical wall located adjacent the staircase 50.

The handrail 550 of staircase 50 is similar in configuration as the handrail 100 shown in FIG. 1 except that handrail 550 is coupled to support stands 54, 56 and a handle 552 of handrail 550 has a greater longitudinal length than the handle 202 of handrail 100. In this exemplary embodiment, the sanitizing module 300 of handrail 550 may travel at a vertical incline across substantially the entire longitudinal length of handle 552 to ensure that at least the substantial entirety of the surface area of an outer surface or exterior 554 of handle 552 may be sanitized by sanitizing module 300. Additionally, it may be understood that the door 10, free-standing rail 30, and staircase 50 shown in FIGS. 1-3, respectively, represent only a few examples of the multitude of applications in which a self-sanitizing handrail similar in configuration to handrails 100, 300, and 500 described above may be incorporated.

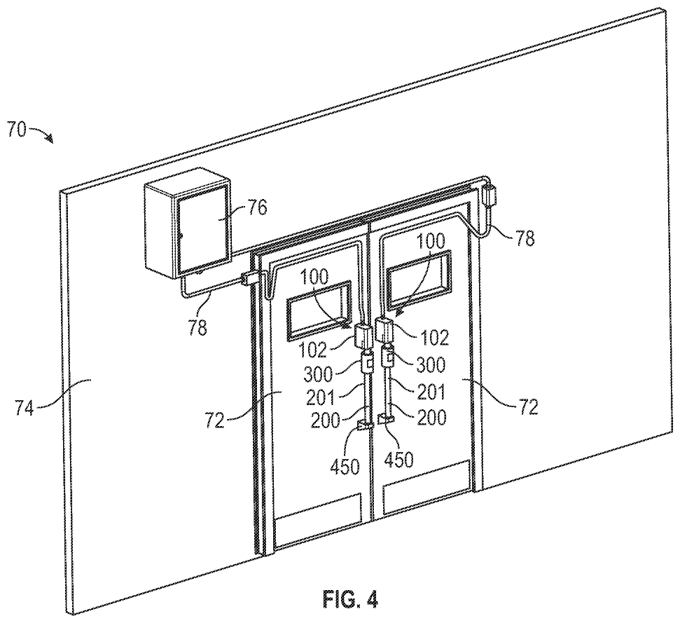

Referring now to FIG. 4, an embodiment of a double door 70 is shown which includes a plurality of self-sanitizing handrails 100. In this exemplary embodiment, double door 70 generally includes a pair of abutting door panels 72 hinged to a wall 74 of an external structure or building, each door panel 72 having a pair of handrails 100 coupled therewith. Particularly, each door panel 72 includes an interior handrail 100 (shown in FIG. 4) located in an interior of the building and an exterior handrail 100 (not shown in FIG. 4) opposite the interior handrail 100 and located on an exterior of the building.

In this exemplary embodiment, electrical power is delivered to each of the handrails 100 of double door 100 via an electrical junction box 76 connected to a power system of the building. For example, junction box 76 may comprise a power supply or transformer that converts a higher voltage (e.g., 110 volt (V), 240V, etc., building electricity) to a lower voltage (e.g., 12V, 24V, etc.) typically required by industrial electronic equipment. It may also be understood that a backup battery module may be located within the junction box 76 in the event of a power outage.

The junction box 76 is electrically connected to the control unit 102 of each of the handrails 100 via a plurality of electrical cables 78 extending therebetween. Particularly, junction box 76 is supported on an interior side of the wall 74 of the building, hidden from view from an exterior of the building for aesthetic purposes and to protect the junction box 76 from the outdoor elements. In this exemplary embodiment, the exterior handrails 100 on the exterior side of wall 74, opposite the interior handrails 100 shown in FIG. 4, may be electrically connected directly to the interior handrails 100 to avoid needing to run electrical cables 78 through the double door 70 and/or wall 74 to electrically connect with the exterior handrails 100. Instead, electrical cables (not shown in FIG. 4) may extend directly between the control panels 102 of the interior handrails 100 and the exterior handrails 100 of double door 70. It may be understood that in other embodiments other signal conveyers besides electrical cables 78 may be utilized to route electrical power to the handrails 100 such as, for example, commercially available door electric power transfers including electric hinges, electric pivots, inductive coupling power transfers, concealed door loops, etc.

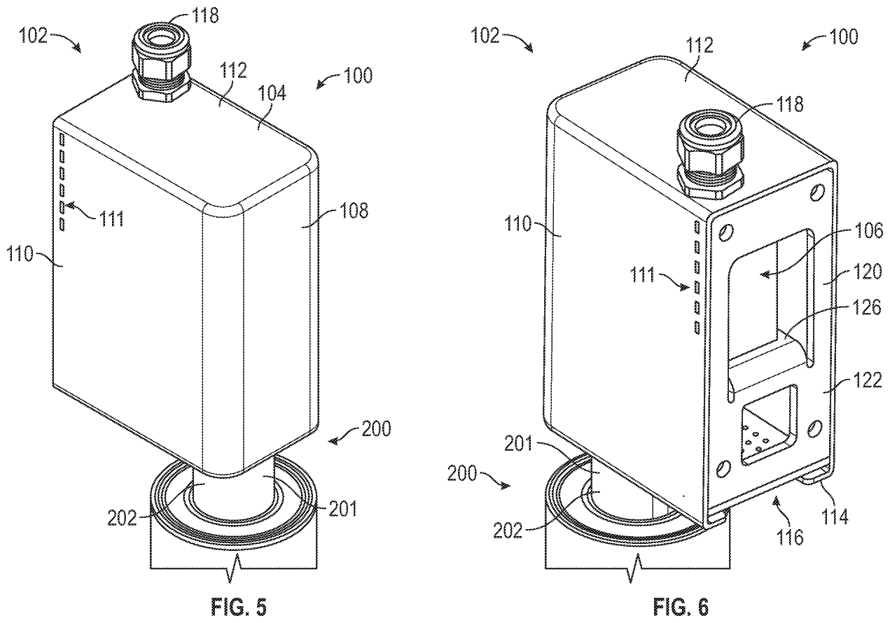

Referring to FIGS. 5-9, an embodiment of the control unit 102 of the handrail 100 of FIG. 1 is shown. In this exemplary embodiment, control unit 102 generally includes an outer covering or housing 104, a mounting bracket 120, an electric motor 150, and a power or drivetrain 180.

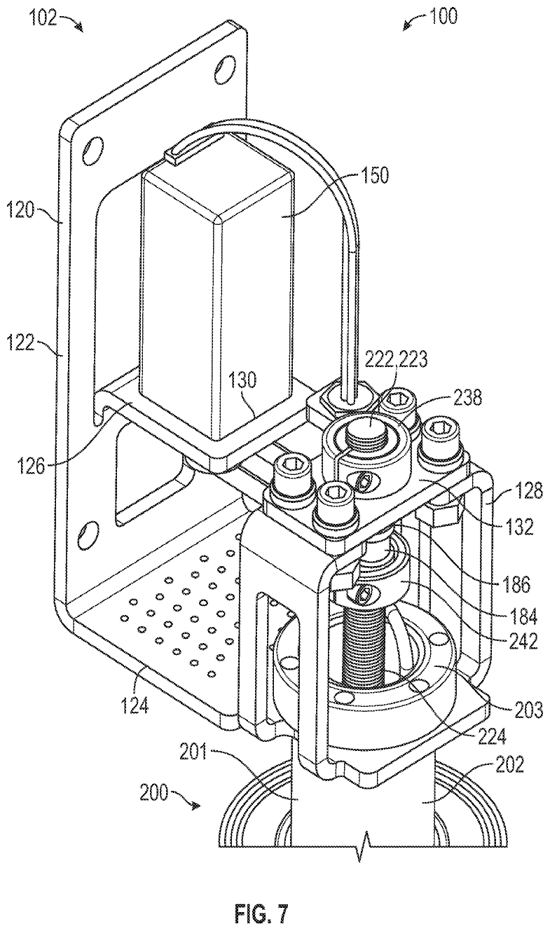

Housing 104 (hidden from view in FIGS. 7-9) of control unit 102 houses the internal components of control unit 102 including, for example, electric motor 150 and drivetrain 180 such that the internal components of control unit 102 are hidden from the view of users of handrail 100. The aesthetic appearance of handrail 100 may be improved by hiding the internal components of control unit 102 within housing 104. Additionally, housing 104 may prevent people from tampering with the internal components of control unit 102.

In this exemplary embodiment, housing 104 particularly includes an internal chamber 106 defined by a front panel 108, a pair of side panels 110, a top panel 112, and a bottom panel 114. It may be noted that housing 104 does not include a rear panel and instead the rear of housing 104 (opposite front panel 108) is open to allow for mounting bracket 120 and components coupled therewith to be inserted into the internal chamber 106 of housing 104. However, a rear surface of housing 104 defined by the rear ends of front panel 108, side panels 110, top panel 112, and bottom panel 114 may sit flush against the external object to which control unit 102 is mounted (e.g., mounting plate 14 of door panel 12) to prevent or at least mitigate the ingress of dust and/or moisture into the internal chamber 106 of housing 104.

Additionally, the bottom panel 114 includes a centrally located slot 116 extending into bottom panel 116 from the rear thereof. Slot 116 may allow for the passage of handle assembly 200 into the internal chamber 106 of housing 104. Further, in this exemplary embodiment, each of the side panels 110 include a plurality of openings 111 located along a rear of the side panel 110 which permit airflow through the internal chamber 106 of housing 104 to cool the internal components of control unit 102 housed therein. In some embodiments, a cooling fan may be mounted within internal chamber 106 to assist with the cooling of the internal components housed therein. Housing 104 may be formed from a light weight plastic in some embodiments (e.g., nylon, polycarbonate, acrylonitrile butadiene styrene (ABS), etc.), but it may be understood the material(s) comprising housing 104 may vary. For example, in other embodiments, housing 104 may comprise a stamped, formed, or cast metallic material such as low carbon steel, stainless steel, aluminum, etc.

Additionally, housing 104 includes an electrical cable gland 118 positioned in the top panel 112 thereof. Cable gland 118 may receive an electrical power cable (not shown in FIGS. 5-9) for providing electrical power to control unit 102. Cable gland 118 may seal the passage of the power cable therethrough to prevent moisture from entering the internal chamber 106 of housing 104. In some embodiments, handrail 100 may be powered by common 110V to 120V alternating current (AC) supplied by a power system of the external structure or building in which the handrail 100 is housed. In other embodiments, handrail 100 may be powered by a DC voltage ranging approximately between 5V and 30V which may be stepped down by a transformer from a higher AC voltage. In some embodiments, handrail 100 may comprise a rechargeable battery module (not shown) installable by an operator of the handrail 100 into the internal chamber 106 of housing 104 for powering the electric motor 150 and/or sanitizing module 300.

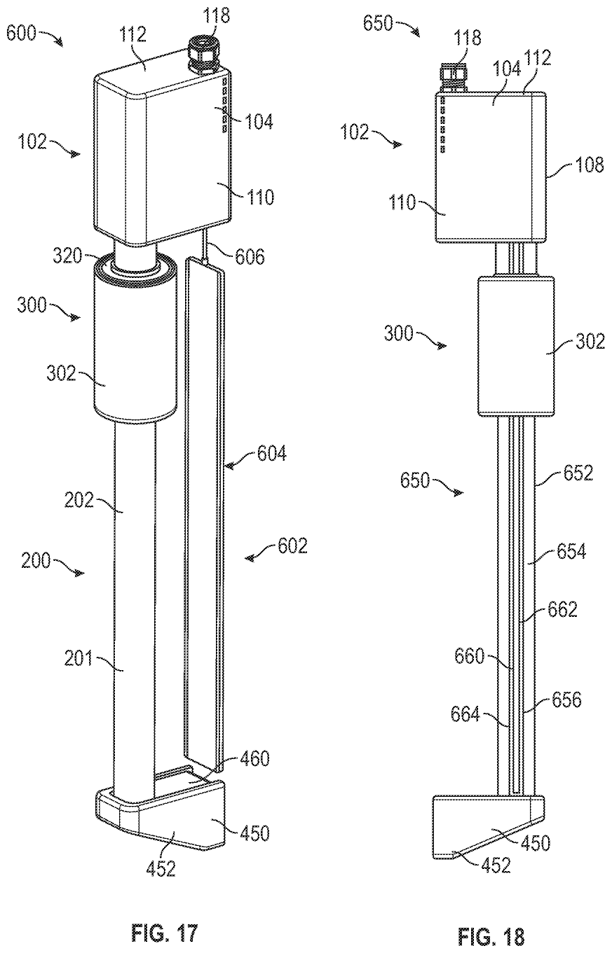

In some embodiments, the electrical power supplied to control unit 102 may be generated by handrail 100 itself rather than from an external source. As an example, referring briefly to FIG. 17 another embodiment of a handrail 600 is shown. Handrail 600 is similar to the handrail 100 shown in FIGS. 5-9 except that handrail 600 includes a solar or photo-voltaic (PV) panel 602 comprising a plurality of solar or PV cells 604. The PV cells 604 of PV panel 602 generate electrical power when exposed to sunlight. The electrical power generated by PV panel 602 may be supplied to the control unit 102 of handrail 600 via a signal conductor or electrical cable 606 which extends between the PV panel 602 and control unit 102.

It may be understood that while at some times handrail 600 may be powered by PV panel 602, at other times handrail 600 may be powered by a separate energy source such as an external electrical system. For example, in some embodiments, PV panel 602 could be used to either meet the immediate power requirements of handrail 600, such that power generated by PV panel 602 is immediately consumed by handrail 600, or alternately, power generated by PV panel 602 could be used to recharge a battery module of handrail 600 that is located internally within control unit 102 or external control unit 102 and connected thereto via one or more electrical cables. In this manner, during hours of high ambient light availability, any energy gathered by PV panel 602 in excess of the amount required to operate handrail 600 would be stored in the battery module, and available to provide power in times when less ambient light is available. Handrail 600 may not include cable gland 118 in embodiments in which PV panel 602 provides all the power required by handrail 600 and/or embodiments in which handrail 600 receives power from another handrail.

Returning to FIGS. 5-9, the mounting bracket 120 supports the internal components of control unit 102 and provides a mount for coupling the control unit 102 to an external structure such as, for example, a door panel, a support stand, etc. In this exemplary embodiment, mounting bracket 120 generally includes a rear panel 122, a bottom panel 124, a motor mount 126, and a handle mount 128. Rear panel 122 of mounting bracket 120 extends along a rear of the control unit 102 and is configured to mount directly to the external structure to which control unit 102 is coupled. Rear panel 122 may include one or more receptacles for receiving one or more removeable fasteners for coupling mounting bracket 120 with the external structure, but it may be understood that a variety of mechanisms may be utilized for coupling mounting bracket 120 with the external structure. In this exemplary embodiment, the motor mount 126 extends from the rear panel 122 towards the front of control unit 102. Motor mount 126 includes a receptacle 130 in which the electric motor 150 of control unit 102 is received to thereby mount (e.g., via one or more fasteners) the electric motor 150 to the mounting racket 120. In this exemplary embodiment, the handle mount 128 extends from the bottom panel 124 of mounting bracket 120 towards the top of control unit 102. A terminal end of the handle mount 128 includes a cradle 132 which is coupled to the handle assembly 200 of handrail 100 such that thrust loads may be transferred between handle assembly 200 and the mounting bracket 120 while permitting rotation between at least a portion of the linear drive 220 of handle assembly 200 and the mounting bracket 120. Cradle 132 may also support thrust loads applied to sanitizing module 300 and transferred through the linear drive 220 to the cradle 132.

Electric motor 150 controls and drives the operation of both the linear drive 220 of handle assembly 200 and the sanitizing module 300. Particularly, electric motor 150 is coupled to the linear drive 220 and is configured to transport the sanitizing module 300 rectilinearly along axis 205 in response to the activation of electric motor 150. Electric motor 150 may be electrically powered by one or more electrical cables (not shown in FIGS. 5-9) which extends from cable gland 118. While in this exemplary embodiment motor 150 is electrically powered, in other embodiments, motor 150 may be powered by another type of power source including pneumatically powered, hydraulically powered, etc.

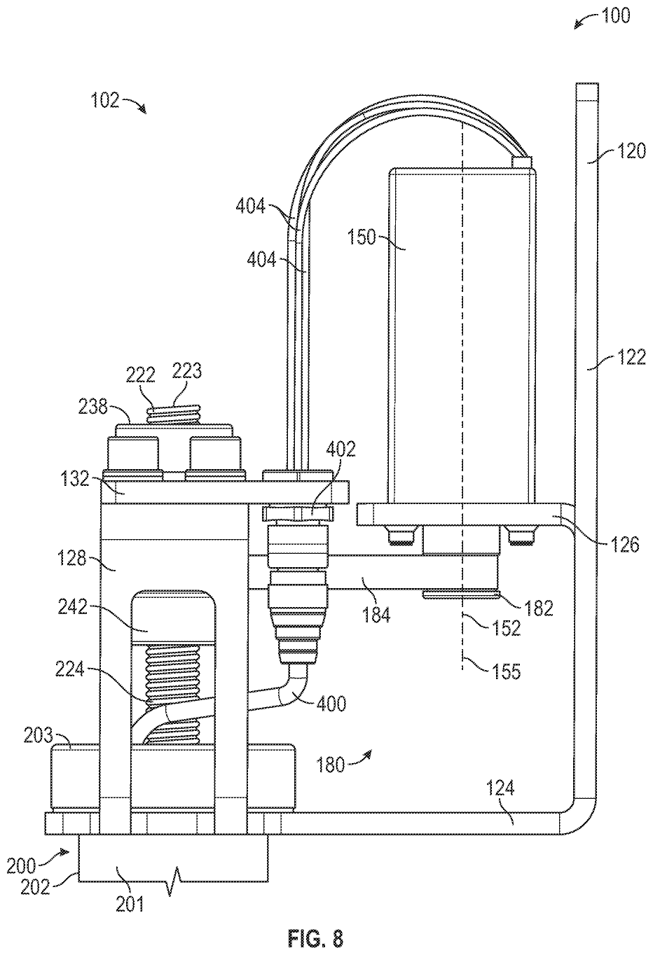

In this exemplary embodiment, electric motor 150 includes a drive or output shaft 152 which is selectably rotatable in both opposing rotational directions about a rotational axis 155 (shown in FIG. 8) of the electric motor 150. In other embodiments, electric motor 150 may comprise a linear motor which does not produce rotation about an output shaft like output shaft 152. As will be described further herein, rotation of the output shaft 152 of electric motor 150 drives the displacement of sanitizing module 300 along the longitudinal length of the handle assembly 200 of handrail 100. In this exemplary embodiment, electric motor 150 comprises a TSM11 series servo motor provided by Applied Motion Products (Morgan Hill, CA) and thus may also be referred to herein as servo motor 150. However, it may be understood that in other embodiments electric motor 150 may comprise other types of motors including stepper motors outfitted with a motor controller and a stepper motor driver

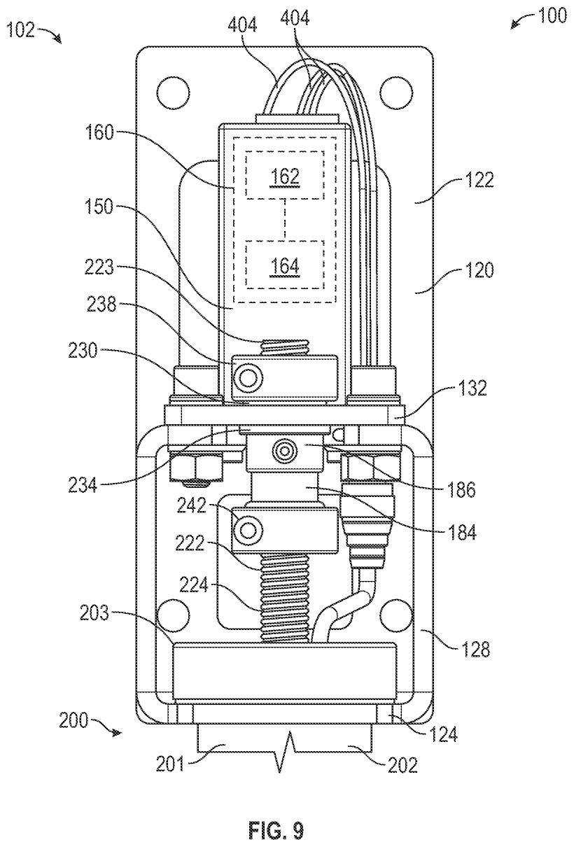

Additionally, in this exemplary embodiment, servo motor 150 comprises a controller 160 (shown in FIG. 9) including both a processor 162 (e.g., a central processing unit (CPU)) and a memory device 164 connected to the processor 162. As will be described further herein, memory device 164 may store instructions for controlling the operation of both linear drive 220 of handle assembly 200 and sanitizing module 300, including the sanitizer 340 thereof. In this manner, controller 160 may automate the operation of handrail 100 such that the components of handrail 100 need not be manually controlled. Particularly, controller 160 is configured to control the activation of the electric motor 150. Additionally, while in this exemplary embodiment controller 160 forms a part of the servo motor 150, it may be understood that in other embodiments, the controller 160 may be separate from the servo motor 150.

In some embodiments, controller 160 additionally includes a wireless communication module connected to the processor 162 and memory device 164. The wireless communications module could allow controller 160 to connect to a nearby communications network such as, for example, a WiFi network. In this manner, the wireless communication module may enable handrail 100 to be controlled remotely over the air, allow for the remote installation of software updates to the controller 160, etc. The wireless communication module may also allow for the remote monitoring of handrail 100 including the amount of time of activation of linear drive 220 and/or sanitizer 340, the monitoring of any errors pertaining to sensors or other equipment of handrail 100 (e.g., errors resulting from overheating, exceeding maximum torque limits, etc.), etc., which may be used in the development of maintenance schedules of handrail 100. It may be understood that controller 160 need not be located within the housing 104 of control unit 102. Instead, for example, controller 160 may be remote of the housing 104 of control unit 102 (e.g., positioned within a junction box similar to junction box 76, etc.) and may control the operation of servo motor 150 and/or sanitizing module 300 remotely via the wireless communication module. In other embodiments, a portion of the controller 160 may be housed within the housing 104 of control unit 102 while another portion of controller 160 may be located remote of the housing 104.

The drivetrain 180 of control unit 102 transfers rotational motion of the output shaft 152 to the linear drive 220 of handle assembly 200 at a desired gear ratio. In this exemplary embodiment, drivetrain 180 generally includes a first or output pulley 182, a drive belt 184, and a second or drive pulley 186. Output pulley 182 is coupled to an outer surface of the output shaft 152 of servo motor 150 such that output pulley 182 rotates in concert with output shaft 152 about the rotational axis 155 of servo motor 150. Drive belt 184 is coupled to the output pulley 182 such that rotation of output pulley 182 is translated into motion of the drive belt 184 about the output pulley 182. Drive belt 184 is additionally coupled to the drive pulley 186 of drivetrain 180 such that motion of drive belt 184 is translated into rotation of drive pulley 186 about a central or longitudinal axis 205 of handle assembly 200 which may also be referred to herein as rotational axis 205. Rotational axis 205 of the handle assembly 200 is laterally offset from the rotational axis 155 of servo motor 150; however, it may be understood that in other embodiments, axes 155, 205 may be aligned with each other.

Referring to FIGS. 9-12, an embodiment of the handle assembly 200 of handrail 100 is shown. In this exemplary embodiment, handle assembly 200 generally includes an outer or exterior tube 201 and the linear drive 220 which is received within the exterior tube 201. Exterior tube 201 forms the exterior handle which is grasped by users of the handrail 100, and thus may also be referred to herein as exterior handle 201. Exterior handle 201 includes a longitudinal first flanged end 203, a longitudinal second flanged end 207 opposite the first flanged end 203, the exterior surface 202 extending between ends 203, 207, and a central passage 204 extending between ends 203, 207 and within which the linear drive 220 is received. In this exemplary embodiment, flanged ends 203, 207 comprise threaded nuts which thread onto opposing longitudinal ends of the exterior handle 201 to tighten the exterior handle 201 against the mounting bracket 120 of control unit 102 and a mounting bracket 460 of handle bracket 450 to form a single rigid structure. However, in other embodiments, flanged ends 203, 207 may be formed integrally with exterior handle 201. In some embodiments, exterior handle 201 may be formed from stainless steel for corrosion resistance such as medical grade stainless steels (e.g., 316 stainless) or other grades of stainless steel. In other embodiments, exterior handle 201 may comprise a carbon steel or other material (e.g., a plastic, glass, etc.) upon which a protective, corrosion resistant coating has been applied.

The first flanged end 203 of exterior handle 201 is received within the control unit 102 and secured to the bottom panel 124 of mounting bracket 120 to thereby restrict relative movement between exterior handle 201 and the mounting bracket 120 of control unit 102. Additionally, the second flanged end 207 of exterior handle 201 is received within handle bracket 450 of handrail 100. In this configuration, external loads applied to exterior handle 201 (e.g., from a user of handrail 100) are transferred to the mounting bracket 120 of control unit 102 and the handle bracket 450 which are attached to an external structure (e.g., a door panel, a pair of support stands, etc.). Thus, external loads applied to exterior handle 201 may be transferred to the external structure through the mounting bracket 120 and bracket handle 450.

In this exemplary embodiment, grooves 209, 211 are formed in the exterior surface 202 of exterior handle 201 proximal the flanged ends 203, 207 thereof, respectively. The bottom panel 124 of mounting bracket 120 is received in the first groove 209 of handle 201 while the handle bracket 450 is received in a second groove 211 of the exterior handle 201. Grooves 209, 211 minimize the tolerances between the exterior handle 201 and the mounting bracket 120 and bracket handle 450 to ensure a desired alignment between these components. It may be understood that in other embodiments exterior handle 201 may not include grooves 209, 211.

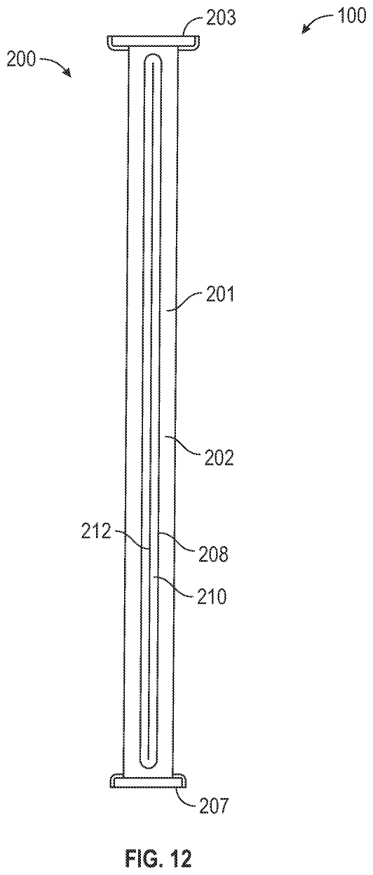

As shown particularly in FIG. 12, exterior handle 201 includes an axially extending slot 208 positioned along a rear of the exterior handle 201. As will be discussed further herein, slot 208 permits sanitizing module 300 to connect with the linear drive 220 of handle assembly 200. Slot 208 extends along a majority of the longitudinal length of the exterior handle 201 to maximize the range of travel of sanitizing module 300. For example, in some embodiments, the longitudinal length of slot 208 may be 80% or greater of the longitudinal length of the exterior handle 201. Slot 208 is positioned along a rear of the exterior handle 201 to keep the slot 208 hidden from the view of users of handrail 100 to aid in the aesthetic appearance of handrail 100.

Additionally, in this exemplary embodiment, handle assembly 200 includes a gasket 210 having a similar size and shape as slot 208 and which is positioned within slot 208. Gasket 210 is formed from a pliable material such as an elastomeric or thermoplastic UV-stable material. Gasket 210 may be secured within slot 208 using adhesive or some other mechanism such as one or more mechanical fasteners and/or a dovetail groove arrangement in which the gasket 210 is received within a dovetail extending along axis 205. Further, gasket 210 includes a narrow slit 212 extending along nearly the entire longitudinal length of the gasket 210. Slit 212 remains normally closed but may elastically deform to provide space sufficient for sanitizing module 300 to extend through the slit 212 as will be discussed further herein. In this manner, slot 208 of exterior handle 201 permits sanitizing module 300 to couple with linear drive 220 while gasket 210 restricts debris from entering the central passage 204 of exterior handle 201 and/or sanitizing light from escaping the central passage 204. In other embodiments, handle assembly 200 may not include gasket 210 and instead a brush seal may be utilized in which a pair of brushes extending along axis 205 are positioned within slot 208 and which may overlap to cover slot 208.

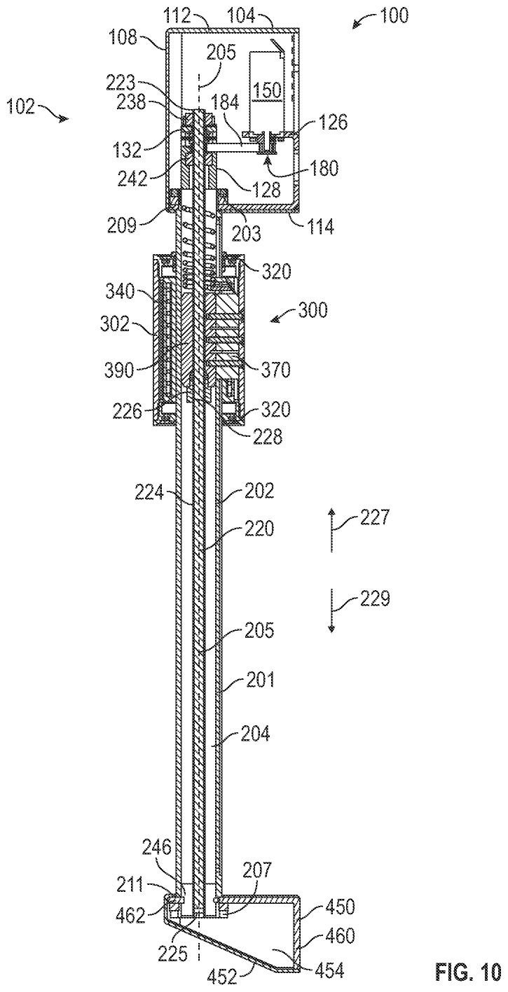

In this exemplary embodiment, the linear drive 220 of handle assembly 200 generally includes a lead screw 222 and a travelling nut 226 positioned along the lead screw 222 where both lead screw 222 and travelling nut 226 each have a central or longitudinal axis that is coaxial with central axis 205. Lead screw 222 extends between a longitudinal first end 223 and a longitudinal second end 225 opposite first end 223. External threads 224 are formed on an outer surface of lead screw 222 while internal threads 228 are formed on an inner surface of screw nut 226. The internal threads 228 of travelling nut 226 are threadably connected or engaged with the external threads 224 of lead screw 222 such that rotation of lead screw 220 about the rotational axis 205 urges travelling nut 226 to rotate in concert about the rotational axis 205. However, as will be described further herein, travelling nut 226 is prevented from rotating about rotational axis 205, and thus rotation of lead screw 222 is translated into linear motion of the travelling nut 226 along the rotational axis 205. In this manner, travelling nut 226 may be displaced linearly in a first direction (indicated by arrow 227 in FIG. 10) in response to rotating lead screw 222 in a first rotational direction. Lead screw 226 may also be displaced linearly in a second direction (indicated by arrow 229 in FIG. 10) in response to rotating lead screw 222 in a second rotational direction opposite the first rotational direction. Lead screw 222 may be rotated by the operation of the drivetrain 180 of control unit 102, where drive pulley 186 is coupled about lead screw 222 whereby lead screw 222 rotates in concert with drive pulley 186 about rotational axis 205.

The first end 223 of lead screw 222 is supported by a flanged sleeve bearing 230 and a thrust bearing 234. The sleeve bearing 230 is seated against a first support surface of the cradle 132 of the handle mount 128 and which extends through an aperture 134 formed in the cradle 132. Sleeve bearing 230 is secured against the first support surface of cradle 132 by a first collar 238 that is threadably coupled to the first end 223 of lead screw 222. Lead screw 222 extends through sleeve bearing 230 and is not threaded to sleeve bearing 230 whereby lead screw 222 may rotate about rotational axis 205 relative to sleeve bearing 230. In this configuration, radial loads and axial or thrust loads in the second direction 229 may be transferred from the lead screw 222, through the sleeve bearing 230, and to the cradle 132 of handle mount 128.

Thrust bearing 234 is seated against a second support surface of cradle 132 opposing the first support surface thereof. Thrust bearing 234 is secured against the first support surface of cradle 132 by a second collar 242 that is threadably coupled to the lead screw 222. Lead screw 222 extends through thrust bearing 234 and is permitted to rotate about rotational axis 205 relative to thrust bearing 234. Axial or thrust loads in the first direction 227 may be transferred from the lead screw 222, through the thrust bearing 234, and to the cradle 132 of handle mount 128.

Additionally, the second end 225 of lead screw 222 is supported by a sleeve bushing 246 received in the exterior handle 202. Particularly, sleeve bushing 246 is coupled to the inner surface of exterior handle 202 at the second end 207 thereof whereby relative rotation between the sleeve bushing 246 and exterior handle 202 is restricted. The second end 225 of lead screw 222 is rotatably positioned in the sleeve bushing 246 whereby radial loads may be transferred from the second end 225 of lead screw 222, through the sleeve bushing 246, and to the exterior handle 202.

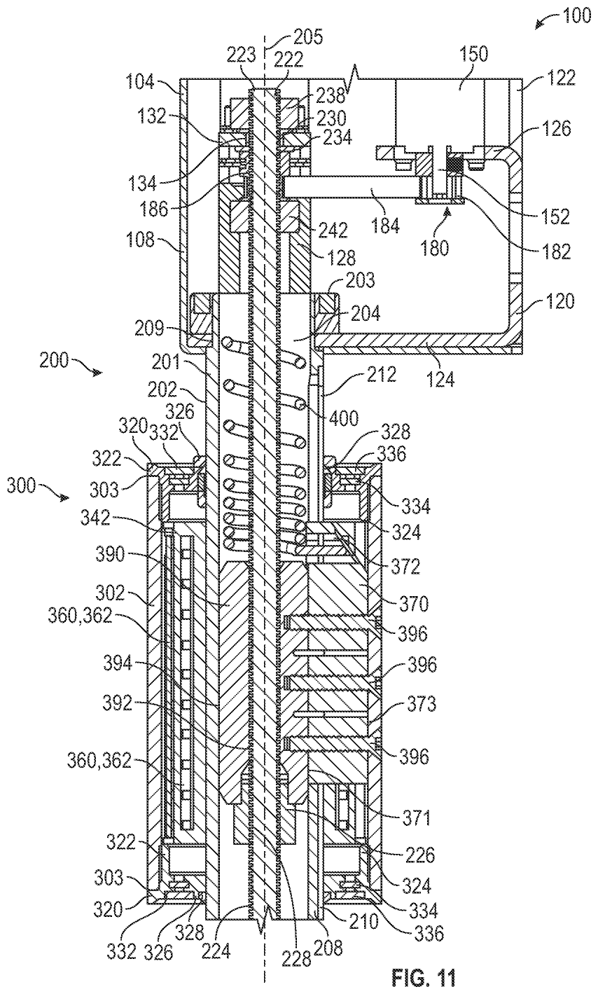

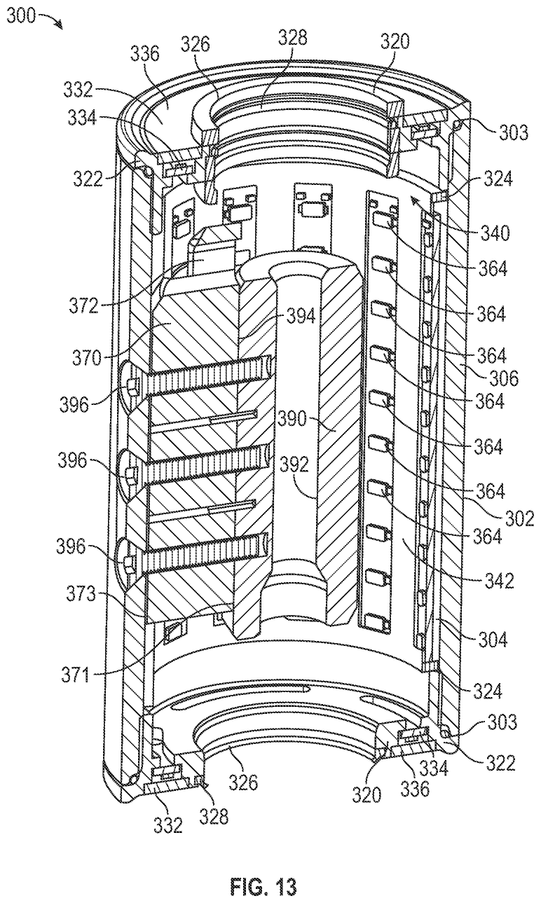

Referring now to FIGS. 11, 13-15, an embodiment of the sanitizing module 300 of handrail 100 is shown. In this exemplary embodiment, sanitizing module 300 generally includes an outer shell or housing 302, a pair of end housings 320, sanitizer 340, a mounting bracket 370, and an inner barrel or nut 390. The outer housing 302 of sanitizing module 300 has a pair of opposed longitudinal ends 303 and a central passage or chamber 304 defined by a generally cylindrical inner surface 306 extending between ends 303. Outer housing 302 physically supports the sanitizer 340 of sanitizing module 300 which is received within the chamber 304 of outer housing 302.

It may be understood that the longitudinal length of sanitizing module 300 relative to the longitudinal length of exterior handle 201 may vary substantially depending on the given application. In some embodiments, the longitudinal length (e.g., the longitudinal length spanning between ends 303 of housing 302) of sanitizing module 300 is less than half of the longitudinal length (e.g., the length spanning between ends 201, 203) of exterior handle 201. However, in other embodiments, the longitudinal length of sanitizing module 300 may be more than half of the longitudinal length of the exterior handle 201.

The end housings 320 of sanitizing module 300 connect to the ends 303 of outer housing 302 to at least partially enclose the chamber 304 of outer housing 302 to prevent or at least mitigate the escape of sanitizing light from chamber 304 to the surrounding environment. In this exemplary embodiment, each end housing 320 generally includes a radially outer (relative to rotational axis 205) hub 322, a radially inner hub 326, and a proximity sensor module 332 positioned radially between hubs 322, 326. The outer hubs 322 of end housings 320 sealingly (e.g., via an O-ring, a metal-to-metal seal, and/or other sealing mechanism) connect to the ends 303 of outer housing 302 to secure the end housings 320 to the outer housing 302 while restricting debris from entering the chamber 304 of outer housing 302 via the annular interfaces formed between outer housing 302 and end housings 320. Chamber 304 also provides a space into which the gasket 210 of handle assembly 200 may deflect into as the gasket 210 is deformed by the travel of sanitizing module 300.

The inner hub 326 of each end housing 320 is positioned directly adjacent or abuts the exterior surface 202 of exterior handle 201 and, in this exemplary embodiment, includes an annular wiper seal 328 formed from a pliable material (e.g., an elastomeric material, etc.) that contacts the exterior surface 202 of exterior handle 201. In this configuration, the inner hubs 326 of sanitizing module 300 slide along the exterior surface 202 of exterior handle 201 when sanitizing module 300 travels linearly along rotational axis 205. Additionally, as sanitizing module 300 travels along rotational axis 205, wiper seals 328 of inner hubs 326 wipe the exterior surface 202 of exterior handle 201, ensuring that debris positioned on exterior surface 202 is prevented from entering the chamber 304 of outer housing 302.

The proximity sensor modules 332 of end housings 320 are located at the longitudinal ends of sanitizing module 300 and detect the presence and/or movement of an external object that is in the path along which sanitizing module 300 is travelling. For example, proximity sensor modules 332 may detect the presence of a user's hand grasping the exterior handle 201. As will be described further herein, signals corresponding to the detection of a user's hand and/or other external objects contacting the exterior handle 201 may be relayed to the controller 160 of control unit 102 as sensor feedback. Based on the signals received from proximity sensor modules 332, controller 160 may cease the operation of servo motor 150 to thereby cease the displacement of sanitizing module 300 along exterior handle 201 to ensure sanitizing module 300 does not inadvertently collide with or contact the external object.

In this exemplary embodiment, each proximity sensor module 332 generally includes a proximity sensor assembly 334 including an annular PCB and a plurality of circumferentially spaced proximity sensors positioned thereon. The proximity sensors of proximity sensor assembly 334 may be equidistantly spaced about the circumference of exterior handle 201 to ensure complete coverage is provided by the proximity sensors.

In some embodiments, each proximity sensor of proximity sensor assembly 334 comprises an infrared emitter, an ambient light sensor, and a communications interface (e.g., an I2C interface). As an example, the proximity sensors of each proximity sensor assembly 334 may comprise the VCNL4020X01 Ambient Light Sensors provided by Vishay Intertechnology, Inc. (Malvern, Pennsylvania). However, it may be understood that a wide variety of differently configured proximity sensors may be utilized in other embodiments.

As an example, in other embodiments, handrail 100 may include a light curtain sensor module having a laser emitter spaced in a circular arrangement around the exterior handle 201 and a receiver also spaced around the exterior handle 201. For example, the laser emitter may be mounted to the control unit 102 while the receiver is mounted to the sanitizing module 300 where feedback data is communicated to the controller 160 from the receiver. Alternatively, both the laser emitter and receiver may be mounted to the control unit 102 and a mirror may be mounted to the sanitizing module 300 for reflecting the laser. In another embodiment, a computer vision camera system may be utilized in which a camera mounted to control unit 102 automatically detects the presence of a foreign object (e.g., a person's hand) that enters the camera's field of view and enters the path of travel of sanitizing module 300. In still another embodiment, handrail 100 may include an accelerometer (e.g., mounted to control unit 102) configured to detect the movement of a door attached to the handrail 100 caused by a user opening/closing the door.

In still another embodiment, controller 160 may determine that a user has grasped the exterior handle 201 by an increase in the resistive torque applied to servo motor 150 that would result from contact between sanitizing module 300 and a user's hand (increasing the resistance to the displacement of sanitizing module 300 along the axis 205). Controller 160 may include a trip which disables servo motor 150 in the event that the resistive torque applied to servo motor 150 exceeds either a predetermined maximum limit or a maximum increase over a predetermined period of time so as to prevent the sanitizing module 300 from injuring a user of handrail 100. The trip of controller 160 may be present in different embodiments of handrail 100 as a fail-safe in the event of a failure of proximity sensor assemblies 334.

Each proximity sensor module 332 additionally includes an annular cover or bezel 336 which encloses and protects the proximity sensor assemblies 334 from the surrounding environment. Bezels 336 may be formed from a transparent material (e.g., glass, acrylic, polycarbonate with sufficient optical transparency, etc.) to permit passage therethrough of the infrared light emitted by the proximity sensors of proximity sensor assembly 334. Additionally, the material of each bezel 336 may be tinted to hide proximity sensor assemblies 334 from the view of users of handrail 100 to improve the aesthetic appearance of handrail 100.

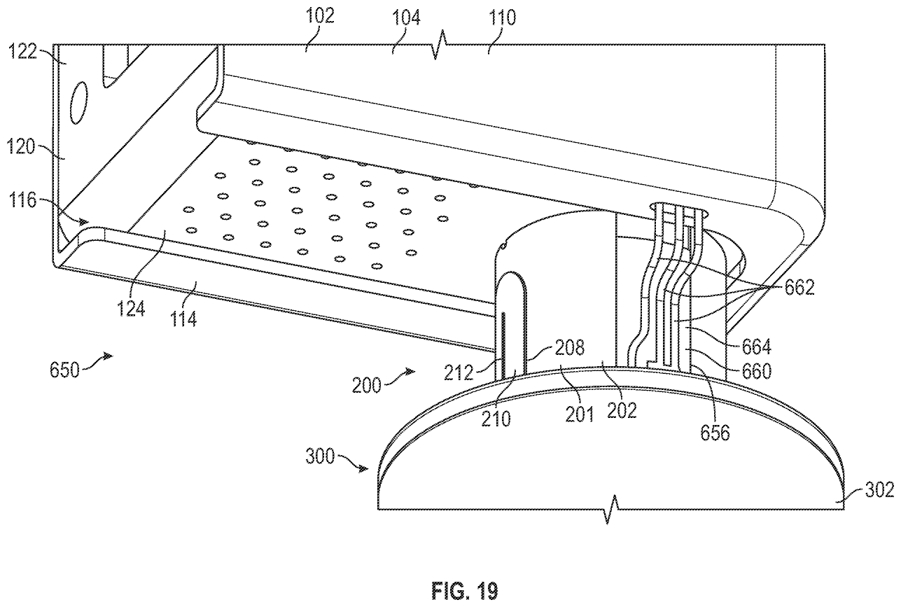

In some embodiments, handrail 100 may include sensors in lieu of or in addition to the proximity sensor assemblies 334 of sanitizing module 300. For example, referring briefly to FIGS. 18, 19, another embodiment of a handrail 650 is shown. Handrail 650 is similar to handrail 100 except that handrail 650 includes an exterior handle 652 having a pair of touch sensor modules 660 positioned on the exterior surface 654 thereof. Particularly, the pair of touch sensor modules 660 are positioned in a pair of grooves 656 formed in the exterior surface 654 of exterior handle 652 and which are spaced approximately 180 degrees apart from each other. It may be understood that in other embodiments handrail 650 may include a single touch sensor module 600 or more than two touch sensor modules 660 positioned at varying circumferential spacings.

Each touch sensor module 660 longitudinally extends the entire length of travel of the sanitizing module 300. Thus, the longitudinal length of each touch sensor module 660 may be 80% or greater of the longitudinal length of the exterior handle 652. Each touch sensor module 660 is configured to detect contact from an external object such as, for example, a user's hand. Additionally, each touch sensor module 660 may determine the longitudinal location along the longitudinal length of touch sensor module 660 where the contact with the touch sensor module 660 takes place. In this manner, touch sensor modules 660 may determine when a user's hand is contacting the exterior handle 652 as well as the location along the longitudinal length of the exterior handle 652 where the contact is occurring. In some embodiments, sanitizing module 300 may not include wiper seal 328 so as not to interfere with the operation of touch sensor modules 660. Signals corresponding to the detection of contact with the exterior handle 652 may be relayed from touch sensor modules 660 to the controller 160 of control unit 102 as sensor feedback such that controller 160 may, for example, cease the operation of servo motor 150 in response to the detection of contact by touch sensor modules 660 with an external object. Additionally, while in this exemplary embodiment, handrail 650 includes both touch sensor modules 660 and the proximity sensor assemblies 334 of sanitizing module 300, in other embodiments, handrail 650 may include only the touch sensor modules 660 and not the proximity sensor assemblies 334.

In this exemplary embodiment, each touch sensor module 660 comprises a membrane potentiometer including an electrically conductive circuit 662 received within a sealed encasement 664. The circuit 662 of each touch sensor module 660 comprises a top circuit and a bottom circuit separated by a spacer. Contact from an external object may bring the top circuit into contact with the bottom circuit thereby producing an electrical output from the circuit 662 that varies based on the location along the longitudinal length of the touch sensor module 660 the contact takes places, allowing for the longitudinal location of the contact to be determined. In some embodiments, each touch sensor module 660 comprises the SoftPot potentiometer provided by Spectra Symbol Corp. (Salt Lake City, UT); however, it may be understood that in other embodiments touch sensor modules 660 may comprise other types of touch sensors which vary in configuration. For example, in other embodiments, touch sensor modules 660 may comprise FSR 408-series force sensing resistors provided by Interlink Electronics® (Irvine, CA), force sensing resistors provided by Sensitronics (Bow, WA), and optical sensors such as the zForce® sensors provided by Neonode® (Stockholm, Sweden).

Returning to FIGS. 10, 12-14, as described above, the sanitizer 340 of sanitizing module 300 is secured within the chamber 304 of outer housing 302 and is configured to sanitize pathogens present on the exterior surface 202 of exterior handle 201 during the operation of handrail 100. In this exemplary embodiment, sanitizer 340 generally includes a C-shaped cradle 342, and a plurality of elongate and circumferentially spaced electrical or electronic light modules 360 coupled to the cradle 342. It may be understood that in other embodiments, the sanitizer 340 may only comprise one or more of the light modules 360 and not the cradle 342.

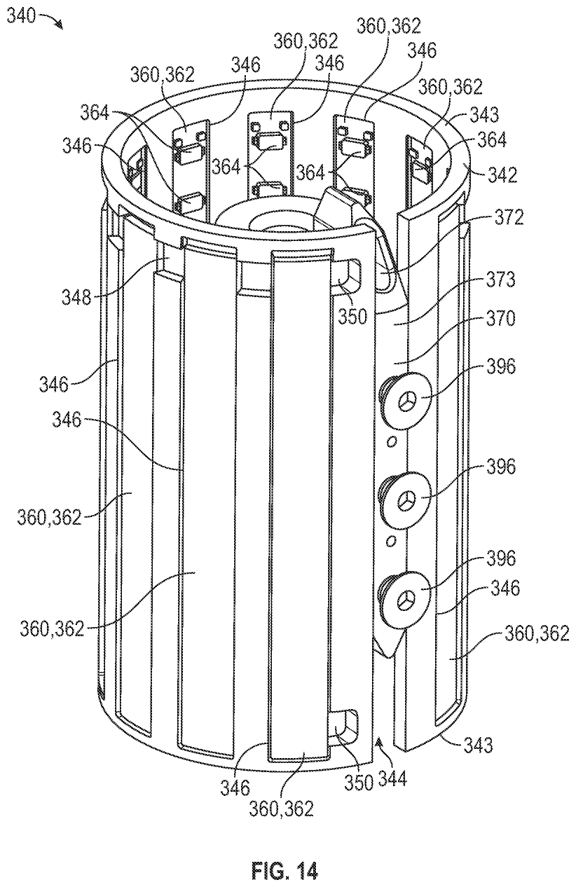

As shown particularly in FIG. 14, in this exemplary embodiment, cradle 342 has a pair of longitudinally opposed ends 343 and includes a through-slot or gap 344 extending between the ends 343. Each end 343 of cradle 342 may contact a terminal end or lip 324 of the outer hubs 322 of sanitizing module 300 to prevent relative axial movement between sanitizer 340 and the outer housing 302 of sanitizing module 300. Additionally, an outer diameter of cradle 342 is sized to be slightly smaller than an inside diameter of outer housing 302 to thereby restrict a central axis of sanitizer 340 from becoming laterally offset from rotational axis 205.

Cradle 342 additionally includes a plurality of circumferentially spaced and elongate receptacles 346 which receive the light modules 360 of sanitizer 340. An annular groove 348 is formed in an outer surface of cradle 342 along one of the ends 343 thereof to provide sufficient space for electrical cables or other signal conductors (not shown) connecting the plurality of light modules 340 together in a “daisy-chain” configuration. Further, cradle 342 includes a pair of radial openings 350 located proximal ends 343 to accommodate the passage of electrical cables or other signal conductors (not shown) extending from sanitizer 340.

Each light module 360 includes an elongate PCB 362 and a plurality of light emitting diodes (LEDs) 364 (only some of which are labeled in FIGS. 11, 13, and 14) spaced along the PCB 362 in a direction that is parallel with rotational axis 205 and each configured to emit sanitizing light. While in this exemplary embodiment a plurality of circumferentially spaced light modules 360 are utilized, in other embodiments, sanitizer 340 may comprise a single light module 360 having a flexible, annular PCB which extends around the circumference of the exterior handle 201, with an array of circumferentially spaced LEDs 364 positioned on the annular PCB.

The LEDs 364 of light modules 360 are configured to project sanitizing light towards the portion of the exterior surface 202 of exterior handle 201 received within the sanitizer 340 of sanitizing module 340. Particularly, LEDs 364 are arranged such that the entire surface area of the axially extending portion of exterior surface 202 encompassed by light modules 360 is irradiated by the sanitizing light emitted by light modules 360. LEDs 364 may comprise commercially available LEDs configured for emitting sanitizing light including, for example, LEDs provided by Klaran (Green Island, NY), Seoul Semiconductor (Seoul, South Korea), Violumas (Freemont, CA), etc. In some embodiments, LEDs 364 comprise UV-C LEDs configured to emit sanitizing light in the UV-C spectrum (e.g., approximately 100 nm to 280 nm). However, in other embodiments, LEDs 364 may be configured to emit sanitizing light in the UV-B (approximately 280 nm to 315 nm) and/or UV-A spectrums (approximately 365 nm to 395 nm). In still other embodiments, LEDs 364 may be configured to emit sanitizing light in the non-UV, blue-wavelengths (e.g., approximately between 400 nm and 420 nm) for the purpose of sanitizing exterior handle 201. While in this exemplary embodiment sanitizing light emitted by LEDs 364 sanitizes the exterior handle 201, in other embodiments, other types of sanitizers may be utilized for sanitizing exterior handle 201. For example, sanitizer 340 may comprise a cloth wipe coupled to one of the end housings 320 of sanitizing module 300 and impregnated with a disinfectant liquid to kill any biological or viral pathogens which come into contact with the cloth wipe. In this manner, the cloth wipe may wipe the exterior surface 202 of exterior handle 201 as the sanitizing module 300 travels along the longitudinal length of handle 201. Sanitizing module 300 may contain a reservoir of disinfectant liquid to continuously supply the cloth wipe with disinfectant during operation. The reservoir may be donut-shaped and located within the chamber 304 of the housing 302 of sanitizing module 300. The reservoir may dispense the disinfectant liquid directly onto the exterior surface 202 of exterior handle 201 and/or onto the cloth wipe via a pumping mechanism. The cloth wipe may be periodically replaced and the reservoir of disinfectant may be periodically refilled by an operator of the handrail 100. In some embodiments, a cloth wipe impregnated or otherwise in contact with a liquid disinfectant may be used to remove any dirt or other debris that has accumulated on the surface 202 of handle 201 which may otherwise interfere with the operation of the sanitizer 340 of sanitizing module 300. For example, the exterior handle 201 may be wiped by the cloth wipe periodically (e.g., once a night), after a predetermined of touches by users o the handrail 100, and/or following some other feedback-driven interval.

In some embodiments, the disinfectant reservoir/pump may be attached to the control unit 102 rather than to the sanitizing module 300. The reservoir may be located within a receptacle formed in housing 104 of control unit 102 and may be conveniently swapped out when empty by an operator of the handrail 100. The disinfectant may be sprayed onto the exterior surface 202 of exterior handle 201 via one or more nozzles attached to the disinfectant pump while the sanitizing module 300 is disposed in a location along exterior handle 201 distal the control unit 102. Following the spraying of the disinfectant, the cloth wipe and/or the wiper seals 328 attached to the sanitizing module 300 may spread or wipe the sprayed disinfectant along the longitudinal length of the exterior handle 201 as the sanitizing module 300 travels linearly therealong.

In some embodiments, one or more warning labels may be positioned on the outside of the sanitizing module 300 or on other parts of handrail 100, such as on an outer surface of the control unit 102. Exterior warning labels may warn users of handrail 100 that sanitizing light is present inside the sanitizing module 300 even though sanitizing light may not be visible from outside the sanitizing module 300 and that the exterior handle 201 is sanitized using sanitizing light to ensure users of handrail 100 that exterior handle 201 is regularly sanitized.

Alternately, in some embodiments, sanitizing module 300 may include colored LEDs coupled to the proximity sensor assemblies 334 which illuminate light in the visible spectrum visible to users of the handrail 100 via bezels 336 of the proximity sensor modules 332. In this manner, colored light may be visible to users of the handrail 100. The colored lights could be a single color to convey to users of handrail 100 that sanitizing module 300 is currently activated. Alternately, multiple colors could be utilized to serve as an external indicator as to the current status of the sanitizing module 300. For example, a first light color could denote that the light modules 360 are currently activated, while a second light color could indicate a problem with the handrail 100, informing an operator of the handrail 100 that the handrail 100 may need to be repaired. In another example, a third light color may indicate that sanitizing module 300 is in a standby mode following the sanitization by sanitizing module 300 of the exterior handle 201.

Referring still to FIGS. 11, 13-15, the mounting bracket 370 of sanitizing module 300 connects the inner barrel 390 with outer housing 302 to thereby mechanically connect sanitizing module 300 with the linear drive 220 of handle assembly 200. Mounting bracket 370 is elongate in shape and is received in the gap 344 of cradle 342. In this exemplary embodiment, mounting bracket 370 has a radially inner (relative to rotational axis 205) end 371, a radially outer end 373 opposite inner end 371, and a radial passage 372 located at a longitudinal end of mounting bracket 370 and extending entirely between the inner end 371 and outer end 373. As will be discussed further herein, radial passage 372 of mounting bracket 372 accommodates the passage of an electrically insulated signal conductor or electrical cable bundle 400 extending from control unit 102 to the sanitizing module 300.

Inner barrel 390 is generally cylindrical and includes a central passage defined by a generally cylindrical inner surface 392 extending between opposed longitudinal ends of inner barrel 390. The mounting bracket 370 is arranged such that outer end 373 of bracket 370 abuts the inner surface 306 of outer housing 302 while the inner end 371 of bracket 370 abuts an outer surface 394 of the inner barrel 390. Additionally, a plurality of fasteners 396 are threaded radially through radial apertures formed in the outer housing 302, mounting bracket 370, and inner barrel 390 to secure both the mounting bracket 370 and inner barrel 390 to the outer housing 302. In this exemplary embodiment, a first longitudinal end and a second longitudinal end of the mounting bracket 370 (each positioned between the inner and outer ends 371, 373) are each tapered to ease the deformation of gasket 210 and thereby reduce resistance to the travel of sanitizing module 300 along axis 205.

In this arrangement, rotation of outer housing 302, mounting bracket 370, and inner barrel 390 about the rotational axis 205 is restricted due to contact between lateral sides of mounting bracket 370 and corresponding lateral edges of the slot 208 of exterior handle 201. Additionally, in this exemplary embodiment, the travelling nut 226 is received in the central passage of inner barrel 390 whereby the inner surface 392 of inner barrel 390 couples to travelling nut 226 (e.g., via threads, one or more set screws, etc.) such that relative rotation between travelling nut 226 is restricted. While in this exemplary embodiment inner barrel 390 and travelling nut 226 comprise two separate members, in other embodiments the travelling nut 226 may be incorporated directly into inner barrel 390 whereby inner barrel 390 is threaded to the lead screw 222. With travelling nut 226 also prevented from rotating about the rotational axis 205, rotation of the lead screw 222 is translated into linear displacement of sanitizing module 300 along rotational axis 205 (e.g., in one of the directions 207, 209) via the threaded engagement formed between the external threads 224 of lead screw 222 and the internal threads 228 of travelling nut 226.

While in this exemplary embodiment the sanitizing module 300 is mechanically coupled to the linear drive 220 via the mounting bracket 370, in other embodiments, sanitizing module 300 may not be mechanically coupled to the linear drive 220. Instead, for example, sanitizing module 300 may be magnetically coupled to the linear drive 220 via one or more magnets positioned within the sanitizing module which are magnetically coupled to one or more magnets of the linear drive 220.

Referring now to FIGS. 7-9, 11, and 15, as described above, cable bundle 400 of handrail 100 electrically connects sanitizing module 300 with control unit 102. Particularly, in this exemplary embodiment cable bundle 400 terminates at an electrical connector 402 which is coupled to the cradle 132 of mounting bracket 120. Additionally, one or more signal conductors or electrical cables 404 extend between the electrical connector 402 and the servo motor 150 to electrically connect the cable bundle 400 with the servo motor 150 and thereby provide signal communication between controller 160 and cable bundle 400.

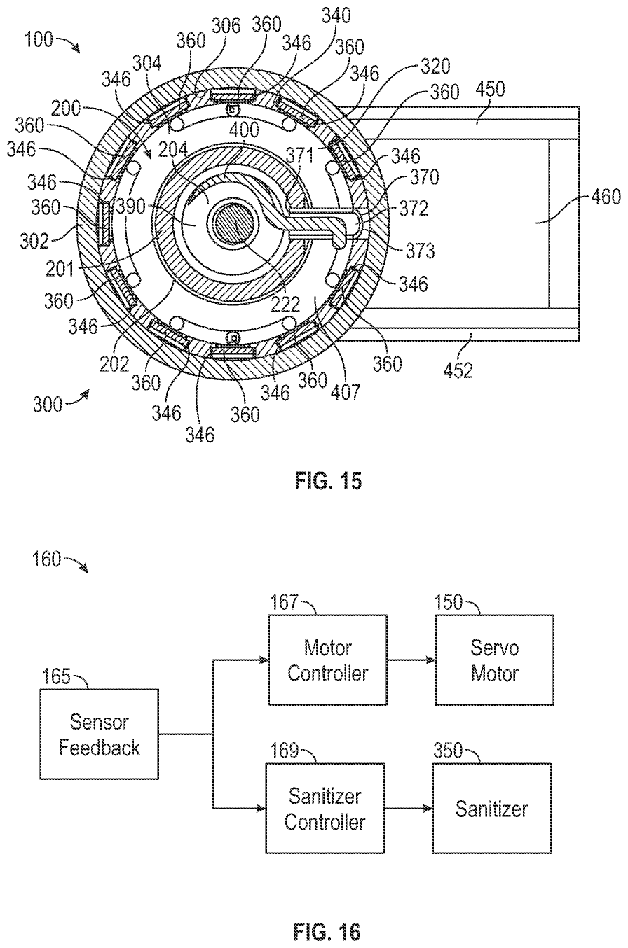

From electrical connector 402, cable bundle 400 extends helically about lead screw 222 within the central passage 204 of exterior handle 201 so as to be hidden from the view of users of handrail 100. Additionally, cable bundle 400 extends from the central passage 204 of exterior handle 201, through the slit 212 formed in gasket 210, and into and through the radial passage 372 of mounting bracket 370 whereby the cable bundle 400 exits the central passage 204 of exterior handle 201 and enters an annulus 407 (shown in FIG. 15) formed between the handle 201 and the outer housing 302 of sanitizing module 300. Cable bundle 400 may be biased to form the helix about lead screw 222 to prevent cable bundle 400 from becoming tangled as sanitizing module 300 is transported along exterior handle 201. From the annulus 407, the individual signal conductors or electrical cables contained in the cable bundle 400 may connect with the various electrical components of sanitizing module 300 including, for example, proximity sensor assemblies 334 and light modules 360. These connections may be made through commercially available electrical connectors, crimp-type connections, direct soldering of the individual electrical cables, and/or other means.

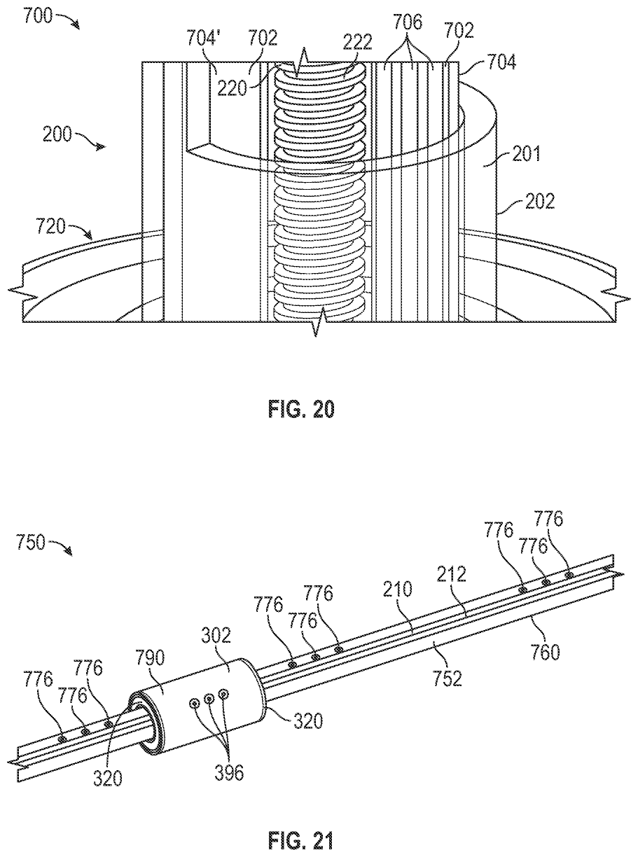

It may be understood that in other embodiments other forms of signal conductors may be used to transmit electrical power and signals between control unit 102 and sanitizing module 300. For example, referring briefly to FIG. 20, another embodiment of a handrail 700 is shown which includes a plurality of elongate electrical rails 702 which extend through the central passage 204 of the exterior handle 201 (shown as transparent in FIG. 20 in the interest of clarity) of handle assembly 200. Each rail 702 comprises an elongate, rigid body 704 and a plurality of electrical conductors 706 formed on the rigid body 704. The rigid body 704 of each rail 702 may be fixed or stationary relative to the exterior tube 201 and at least a portion of each rigid body 704 may comprise PCB along which the electrical conductors 706 are positioned. A terminal end of each electrical conductor 706 may contact a spring-loaded electrical contact or connector of a control unit of the handrail 700 which is similar in configuration to the control unit 102 described above. Additionally, handrail 700 includes a sanitizing module 720 similar in configuration to sanitizing module 300 except that a mounting bracket of the sanitizing module 720 includes a plurality of spring-loaded electrical contacts or connectors which slidably contact the electrical conductors 706 of rails 702 and thereby electrically connect with rails 702.

Referring now to FIG. 10, as described above, the handle bracket 450 secures the second end 207 of handle assembly 200 to the external structure to which handrail 100 is mounted. In this exemplary embodiment, handle bracket 450 includes an outer covering or housing 452 and the L-shaped mounting bracket 460. Housing 452 defines an internal chamber 454 in which the second end 207 of exterior handle 201 is received and thereby hides the second end 207 of exterior handle 201, as well as mounting bracket 460, from the view of users of handrail 100. Additionally, housing 452 may be of similar construction as the housing 104 of control unit 102.

The mounting bracket 460 of handle bracket 450 provides a mount for coupling the second end 207 of exterior handle 201 to an external structure (e.g., a door panel, a support stand, etc.). Particularly, mounting bracket 460 includes an aperture 462 through which the second end 207 of exterior handle 201 extends, coupling the second end 207 of exterior handle 201 to the mounting bracket 460. Additionally, it may be understood that the mounting bracket 120 of control unit 102 may also be referred to herein as first mounting bracket 120 while the mounting bracket 460 of handle bracket 450 may also be referred to herein as second mounting bracket 460.

Referring now to FIGS. 9-11, 16, a schematic block diagram of the controller 160 is shown in FIG. 16. In this exemplary embodiment, controller 160 includes a sensor feedback module 165, a motor controller module 167, and a sanitizer controller module 169. Modules 165, 167, and 169 of controller 160 may be embodied in software stored in the memory device 164 of controller 160 and may be executable via the processor 162 of controller 160. In some embodiments, motor controller module 167 may comprise an encoder (in embodiments where electric motor 150 comprises a servo motor) and may store a thread pitch of the lead screw 222 of linear drive 220 whereby motor controller module 167 may track and monitor a linear position of the sanitizing module 300 along axis 205 at all times.

In this exemplary embodiment, the motor controller module 167 and sanitizer controller module 169 each receive sensor feedback from the sensor feedback module 165. For example, sensor data from proximity sensor assemblies 334, touch sensor modules 660, and/or other sensors may be provided to motor controller module 167 and sanitizer controller module 169 as sensor feedback. Motor controller module 167 provides a control output to the servo motor 150 of control unit 102 for controlling the operation of linear drive 220 while sanitizer controller module 169 provides a control output to the sanitizer 340 of sanitizing module 300.

Modules 167, 169 of controller 160 may control the operation of servo motor 150 and sanitizer 340 in accordance with one or more predefined routines stored in the memory device 164 of controller 160. For example, in an exemplary embodiment, sanitizing module 300 may travel rectilinearly along axis 205 at a predetermined speed back-and-forth along the exterior handle 201 continuously. Particularly, sanitizing module 300 may travel continuously between a first longitudinal position proximal the first end 203 of exterior handle 201 and a second, opposed second longitudinal position proximal the second end 203 of exterior handle 201. In this exemplary embodiment, the sanitizing module 300 would only stop travelling along axis 205 in the event that a user's hand or other object is detected in the path of travel of sanitizing module 300 by proximity sensor assemblies 334. Controller 160 may also deactivate light modules 360 in response to detecting the presence of a user's hand in the path of travel of sanitizing module 300 to ensure the user's hand is not irradiated by sanitizing light generated by light modules 360.

This speed of sanitizing module 300 may be calculated in accordance with a predetermined sanitization confidence interval. Confidence intervals such as, for example, 95%, 99%, 99.9%, 99.99% are commonly established in the field of statistical analysis, and such confidence intervals could be selected on an application specific basis, and would then in turn determine the time irradiation time required by sanitizing module 300 (and thus the speed of sanitizing module 300 along axis 205) at a given location along the exterior handle 201 in order to achieve the required level of confidence of the sanitization. The required time to achieve a given confidence interval may depend, among other factors, on the wavelength and intensity of the sanitizing light produced by the light modules 360 of sanitizing module 300. The controller 160 may track the electrical power consumed by the light modules 360 along with the total amount of time light modules 360 have been activated during their lifespan. This data, in comparison with benchmark data either provided by the manufacturer of the LEDs 364, or from independent emitter effectiveness and life expectancy testing, could be used to adjust the required irradiation time based on the total cumulative operating time of the LEDs 364, and knowing that over their lifespan, there is some degradation in performance and light output.

In this exemplary embodiment, controller 160 continuously logs data on travel speed of sanitizing module 300 with respect to time. This data can be processed by controller 160 to determine how long the light modules 360 have been positioned over a given point along the exterior handle 201. Based on this data, if the sanitizing module 300 has been unable to move for some period of time (due to continuously detecting an object in the path of the sanitizing module 300), at some point, the controller 160 may calculate that a given area of the exterior handle 201 has been continuously irradiated with sanitizing light for more than the minimum time required to ensure sufficient sanitation of that area. At that point, the controller 160 may deactivate the sanitizer 340 (including light modules 360) of sanitizing module 300 in order to conserve power. Once the sanitizing module 300 resumes its linear travel along axis 205, the sanitizer 340 may resume functioning.

In another exemplary embodiment, sanitizing module 300 may remain parked at a predefined parked position or location along the axis 205, such as at a bottom or a top of the exterior handle 201, until a user touches the exterior handle 201. This embodiment would thus incorporate the touch sensor modules 660 described above to allow the controller 160 to determine when and where along the longitudinal length of exterior handle 201 the handle 201 has been touched by the user.

In this exemplary embodiment, based on the feedback data provided by the touch sensor modules 660, controller 160 may operate servo motor 150 and the sanitizer 340 of sanitizing module 300 to sanitize the specific areas of exterior handle 201 that have been touched. Once the sanitizing of the touched area is completed, the sanitizing module 300 returns to its parked position until the next time controller 160 determines that a person has touched the exterior handle 201. This exemplary embodiment may be employed in applications in which the exterior handle 201 is touched relatively infrequently. However, at times when the exterior handle 201 is touched rapidly by a large number of users, the controller 160 may switch the operational mode of the handrail 100 such that the sanitizing module 100 travels continuously and rectilinearly along the exterior handle 201, stopping only to avoid contacting users' hands. Additionally, a speed of the sanitizing module 300 along axis 205 may be adjusted by controller 160 based on the frequency at which exterior handle 201 is touched by users thereof such that, for example, the speed of sanitizing module 300 is increased in response to an increase in frequency of physical contact between users and the exterior handle 201. The controller 160 may modulate the intensity of the sanitizing light emitted by light modules 360 in accordance with changes in speed of sanitizing module 300, where an increase in speed may result in an increase in the intensity of the sanitizing light emitted by modules 360 in order to maintain a desired confidence interval of sanitization of exterior handle 201. It may also be understood that an operator of the handrail may periodically wipe down the exterior handle 201 to remove any dirt or other debris that has accumulated on the surface 202 of handle 201 which may otherwise interfere with the operation of the sanitizer 340 of sanitizing module 300.

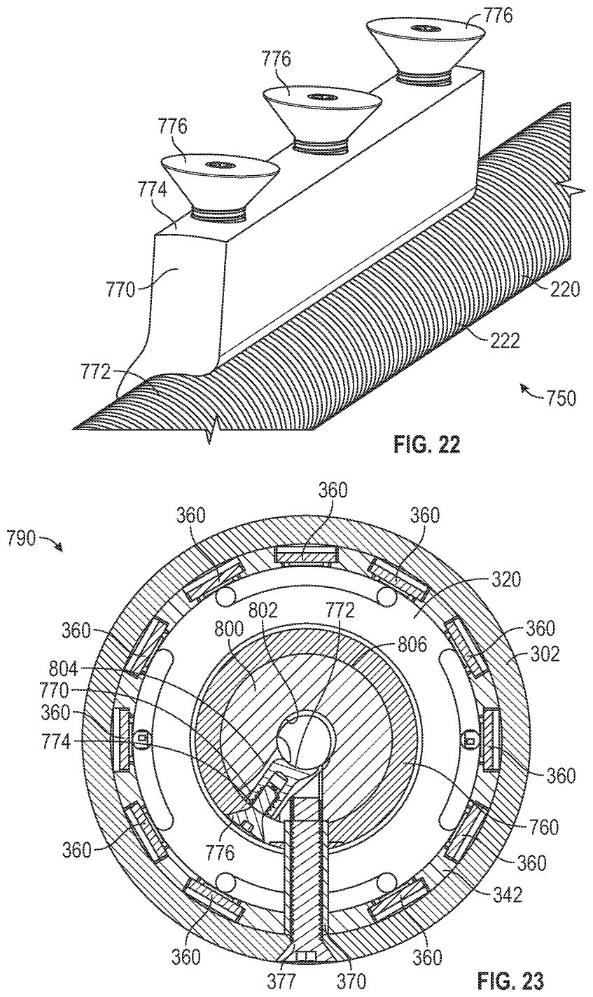

Referring now to FIGS. 21-23, another embodiment of a handrail 750 is shown configured for applications in which a handle assembly 752 of the handrail 750 has a relatively long length, such as in applications in which handrail 750 is incorporated into a relatively large free-standing rail or staircase. Particularly, in this exemplary embodiment, the handle assembly 752 of handrail 750 includes an exterior handle 760 which is supported by a plurality of internal supports 770 spaced at regular intervals along the longitudinal length of the exterior handle 760. As shown particularly in FIG. 22, each internal support 770 is elongate in shape and includes a radially inner end defined by a curved support surface 772 and a radially outer end 774 opposite the support surface 772.

The support surface 772 of each internal support 770 contacts the lead screw 222 of the linear drive 220 of handrail 750 such that radially directed loads (e.g., bending loads applied to lead screw 222) are transferred from the lead screw 222 to the internal support 770. Additionally, the outer end 774 of each internal support 770 is coupled to the exterior handle 760 of handrail 750 via a plurality of separate fasteners threadably received in receptacles formed in the outer end 774 of the internal support 770. In this manner, loads transferred from the lead screw 222 to the internal support 770 may be in-turn transferred from the internal support 770 to the exterior handle 760. From the exterior handle 760, the loads transferred from internal supports 770 may be transferred from the handle 760 to the mounting brackets 120, 460 of the handrail 750.

Handrail 750 additionally includes a sanitizing module 790 similar to sanitizing module 300 described above except that sanitizing module 790 includes an inner barrel 800 having a central passage that is at least partially threaded to the lead screw 222 of linear drive 220 and a radial slot 804 extending radially outwards from the central passage 802 to an outer surface 806 of the inner barrel 800. Radial slot 804 is sized to fit the internal supports 770 as shown particularly in FIG. 23 such that internal supports 770 may pass through sanitizing module 790 as the sanitizing module 70 travels along the longitudinal length of exterior handle 760. Additionally, similar to inner barrel 390 described above, inner barrel 800 is coupled to mounting bracket 370 by fasteners 396 which prevents relative rotation between inner barrel 800 and lead screw 222.

As described above, the internal supports 770 transfer bending loads from the lead screw 222 to the exterior handle 760 where they may be transferred to mounting brackets 120, 460 of the handrail 750 so as to provide adequate structural support to the handle assembly 752 of handle 750 even in applications in which exterior handle 760 has a significantly large longitudinal length (e.g., longitudinal lengths in excess of 20 feet (ft)).

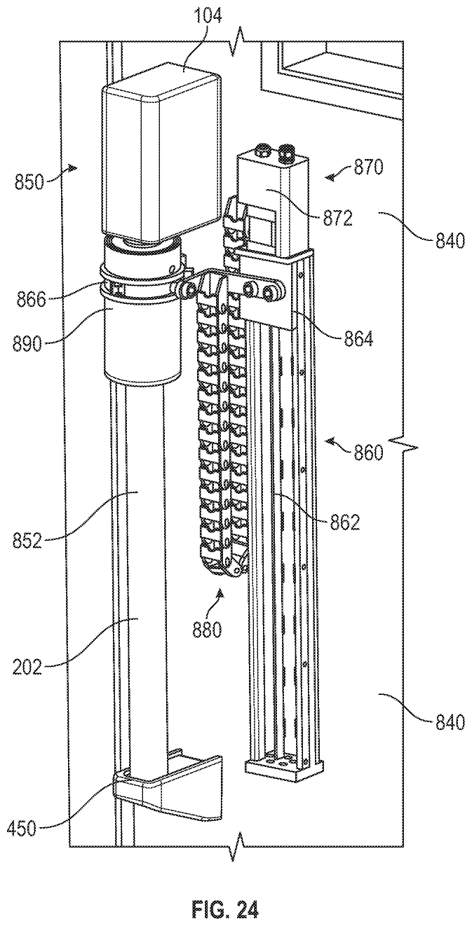

Referring now to FIG. 24, another embodiment of a handrail 850 mounted to a door panel 840 is shown having an external linear drive 860 which extends along, but offset from, a central axis of an exterior handle 852 of the handrail 850. Particularly, in this exemplary embodiment, handrail 850 includes the exterior handle 852 in the form of a solid rod not having an internal passage or chamber, the external linear drive 860 located external the exterior handle 852, a control unit 870 connected to an end of the linear drive 860, and a cable tray 880 also located external the exterior handle 852 and positioned between the exterior handle 852 and the linear drive 860.

In this exemplary embodiment, the linear drive 860 generally includes a rotatable lead screw 862, a carriage 864 coupled to the lead screw 862, and an L-shaped bracket 866 coupled to a sanitizing module 890 of the handrail 850. The lead screw 862 of linear drive 860 is mechanically connected to an electric motor 872 (e.g., a servo motor, etc.) of the control unit 870 which may be configured similarly as electric motor 150 described above and thus may include a controller similar in configuration to controller 160 also described above. Rotation of an output shaft of electric motor 872 produces a corresponding rotation of lead screw 862. Additionally, rotation of lead screw 862 causes carriage 864 to travel linearly along a longitudinal axis or length of the lead screw 862, where the direction of linear travel of carriage 864 corresponds to a rotational direction of the lead screw 862. While a lead screw 862 is utilized in this embodiment, it may be understood that in this and other embodiments (including embodiments with internal linear drives) a chain drive, belt-drive, a linear motor, and/or other types of linear actuators may be used in lieu of the lead screw 862.

The carriage 864 of linear drive 860 is coupled to the sanitizing module 890 of handrail 850 through the bracket 866. In this configuration, sanitizing module 890 travels linearly along a longitudinal axis or length of the exterior handle 852 in concert with the carriage 864 which travels similarly along the longitudinal length of lead screw 862.

Sanitizing module 890 may operate similarly as the sanitizing module 300 described above, and thus may sanitize the exterior surface 202 of exterior handrail 852 using sanitizing light produced by a plurality of light modules 860. Additionally, in this exemplary embodiment, cable tray 880 receives one or more electrical cables extending between sanitizing module 890 and the control module 870. Cable tray 880 arranges the one or more cables such that they do not interfere with the linear travel of the carriage 864 of linear drive 860 and sanitizing module 890.

While exemplary embodiments have been shown and described, modifications thereof can be made by one skilled in the art without departing from the scope or teachings herein. The embodiments described herein are exemplary only and are not limiting. Many variations and modifications of the systems, apparatus, and processes described herein are possible and are within the scope of the invention. For example, the relative dimensions of various parts, the materials from which the various parts are made, and other parameters can be varied. Accordingly, the scope of protection is not limited to the embodiments described herein, but is only limited by the claims that follow, the scope of which shall include all equivalents of the subject matter of the claims. Unless expressly stated otherwise, the steps in a method claim may be performed in any order. The recitation of identifiers such as (a), (b), (c) or (1), (2), (3) before steps in a method claim are not intended to and do not specify a particular order to the steps, but rather are used to simplify subsequent reference to such steps.

1. A self-sanitizing handrail system, comprising:
an elongate handle extending along a central axis and comprising an exterior surface graspable by users of the handrail system;
an annular sanitizing module positioned around the handle and comprising an outer housing and a sanitizer positioned in the housing and configured to sanitize by a sanitizing light emitted by the sanitizer pathogens located on the exterior surface of the handle;
a linear drive extending along the central axis of the handle and coupled to the sanitizing module; and
a motor coupled to the linear drive and configured to transport the sanitizing module rectilinearly along the central axis of the handle in response to activation of the motor.

2. The handrail system of claim 1, wherein the linear drive comprises a lead screw rotatably coupled to the motor, and a travelling nut coupled to the sanitizing module and received within an interior passage of the handle, wherein the travelling nut and sanitizing module are each transportable linearly along the central axis of the handle in response to rotation of an output shaft of the motor.

3. The handrail system of claim 2, further comprising a control unit connected to a longitudinal end of the handle, wherein the control unit comprises an outer housing in which the motor is received, and wherein a longitudinal end of the lead screw extends into the outer housing of the control unit.

4. The handrail system of claim 1, wherein:
the handle comprises an elongate slot which extends radially through the handle; and
the sanitizing module comprises a mounting bracket coupled to the linear drive and which extends radially through the elongate slot of the handle.

5. The handrail system of claim 4, wherein the sanitizing module comprises an annular outer housing connected to a radially outer end of the mounting bracket, and an inner barrel received in an interior passage of the handle and coupled to a radially inner end of the mounting bracket, and wherein the sanitizer is received in the outer housing of the sanitizing module and extend arcuately around the exterior surface of the handle.

6. The handrail system of claim 1, wherein the sanitizer of the sanitizing module includes one or more light modules configured to emit the sanitizing light directed against the exterior surface of the handle to sanitize pathogens located on the exterior surface

7. A self-sanitizing handrail system, comprising:
an elongate handle extending along a central axis and comprising an exterior surface graspable by users of the handrail system; and
an annular sanitizing module positioned around the handle and transportable linearly along the central axis of the handle, wherein the sanitizing module comprises a sanitizer including a light module configured to emit sanitizing light directed against the exterior surface of the handle to sanitize pathogens located on the exterior surface.

8. The handrail system of claim 7, wherein the sanitizing module comprises:
an annular outer housing which extends circumferentially around the exterior surface of the handle and forms a chamber in which the sanitizer is received; and
wherein the sanitizer comprises an annular cradle which physically supports a plurality of the light modules which are circumferentially spaced about the exterior surface of the handle.

9. The handrail system of claim 7, wherein the light module comprises a printed circuit board (PCB) and a light emitting diode (LED) coupled to the PCB and configured to emit the sanitizing light directed against the exterior surface of the handle.

10. The handrail system of claim 7, wherein the sanitizing module comprises a proximity sensor module configured to detect the presence of a person's hand in a path of travel of the sanitizing module.

11. The handrail system of claim 7, further comprising a linear drive extending through an interior passage of the handle and coupled to the sanitizing module and configured to transport the sanitizing module rectilinearly along the central axis of the handle.

12. The handrail system of claim 7, further comprising a control unit coupled to the handle and comprising a motor coupled to the sanitizing module, wherein the motor is configured to transport the sanitizing module linearly along the central axis of the handle, and a controller configured to control the activation of the motor.

13. The handrail system of claim 12, wherein the controller comprises a sanitizer controller module configured to control the operation of the light module of the sanitizer.

14. A self-sanitizing handrail system, comprising:
an elongate handle extending along a central axis and comprising an exterior surface graspable by users of the handrail system,
an annular sanitizing module positioned around the handle and comprising an outer housing and a sanitizer positioned in the housing of the housing and configured to sanitize by a sanitizing light emitted by the sanitizer pathogens located on the exterior surface of the handle; and
a control unit coupled to the handle and comprising a motor coupled to the sanitizing module, wherein the motor is configured to transport the sanitizing module linearly along the central axis of the handle in response to activation of the motor, and a controller configured to control the activation of the motor.

15. The handrail system of claim 14, wherein the handle comprises a touch sensor module positioned on the exterior surface of the handle and connected to the controller, and wherein the controller is configured to determine a longitudinal contact location along the handle at which a person's hand contacts the handle in response to the person's hand contacting the touch sensor module at the contact location.

16. The handrail system of claim 15, wherein the controller is configured to transport the sanitizing module to the contact location to sanitize the contact location in response to the person's hand contacting the touch sensor module at the contact location.

17. The handrail system of claim 14, wherein the sanitizing module comprises a proximity sensor module that is connected to the controller and configured to detect the presence of a person's hand in a path of travel of the sanitizing module.

18. The handrail system of claim 14, wherein the controller is configured to continuously transport the sanitizing module between a first position located at a first end of the handle, and a second position located at a second end of the handle longitudinally opposite the first end.

19. The handrail system of claim 14, further comprising a linear drive extending through an interior passage of the handle and coupled to the sanitizing module and configured to transport the sanitizing module rectilinearly along the central axis of the handle.

20. The handrail system of claim 14, wherein the sanitizer of the sanitizing module includes one or more light modules configured to emit the sanitizing light directed against the exterior surface of the handle to sanitize pathogens located on the exterior surface.
